(19) United States
(12) Patent Application Publication
(10) Pub. No.: US 2021/0308308 A1
(43) Pub. Date: Oct. 7, 2021

(54) SYRINGE AND KIT FOR INTRAVESICAL USE

(71) Applicant: Farco-Pharma GmbH, Koln (DE)

(72) Inventors: Frank SODHA, Koln (DE); Andreas KÜHBACHER, Koln (DE)

(21) Appl. No.: 17/145,679

(22) Filed: Jan. 11, 2021

(30) Foreign Application Priority Data
Apr. 3, 2020 (DE) 1020200021360
Apr. 23, 2020 (DE) 1020201110804

(51) Int. Cl. A61L 2/20; A61M 5/315; A61K 31/216; A61K 9/00

(52) U.S. Cl. CPC A61L 2/20; A61M 5/315; A61M 2205/0205; A61K 9/0019; A61L 2202/23; A61K 31/216

(57) ABSTRACT

The present invention relates to a method for producing a sterile oxybutynin-comprising composition in a preferably ready-to-use piston syringe or for producing a preferably ready-to-use sterile piston syringe filled with an oxybutynin-comprising composition as well as a sterile oxybutynin-comprising composition and piston syringe as such.

### CROSS-REFERENCE TO RELATED APPLICATION

This application claims the benefit of German Application No. DE 10 2020 002 136.0 filed on Apr. 3, 2020, and German Application No. DE 10 2020 111 080.4 filed on Apr. 23, 2020, and incorporates all by reference herein, in its entirety.

### BACKGROUND OF THE INVENTION

The present invention relates to the medical-technical field of sterilization or germ reduction of a special composition which comprises oxybutynin hydrochloride as active ingredient and which is inserted or is present in a piston syringe, especially a ready-to-use one.

Especially the present invention relates to a method for producing a sterile oxybutynin-comprising composition in a preferably ready-to-use piston syringe or for producing a preferably ready-to-use, sterile piston syringe filled with an oxybutynin-comprising composition.

The present invention also relates to a sterile oxybutynin-comprising composition as such, as it is used especially for use in the prophylactic or therapeutic treatment of neurogenic bladder dysfunction, especially by instillation into the urinary bladder.

Furthermore, the present invention also relates to a piston syringe filled with the sterile oxybutynin-comprising composition as such, wherein the oxybutynin-comprising composition comprised in the piston syringe, including the piston syringe, has been vapor sterilized according to the inventive method.

Furthermore, the present invention also relates to a packaging unit which comprises at least one piston syringe according to the invention, wherein the piston syringe is inserted into a packaging or is present in a packaging.

Furthermore, the present invention also relates to corresponding kits based on the piston syringe according to the invention or the oxybutynin-comprising composition according to the invention.

The present invention also relates to the use of a piston syringe in a method for producing a sterile oxybutynin-comprising composition as well as the use of vapor sterilization in a method for producing a sterile oxybutynin-comprising composition in a preferably ready-to-use piston syringe.

The active ingredient oxybutynin (synonymously also referred to as 4-diethylaminobut-2-inyl-2-cyclohexyl-2-hydroxy-2-phenyl ethanoate) or oxybutynin hydrochloride is an anticholinergic which is used for the treatment of bladder malfunction or dysfunctions of the urinary bladder, especially those bladder dysfunctions which are associated with or caused by detrusor hyperactivity and/or detrusor sphincter dyssynergy.

Neurogenic bladder dysfunction or neurogenic bladder voiding disorders often represent a pathological situation of the bladder and the anatomical structures involved in urine storage and excretion that requires treatment. Neurogenic bladder dysfunction is especially a dysfunction of the bladder or the relevant anatomical structures that occurs as a result of a malfunction or injury of the nervous system, for example as a result of spinal cord injury, spina bifida (“open back”), diabetes, multiple sclerosis, stroke or Parkinson's disease. Patients suffering from bladder dysfunction can thereby suffer a great deal, especially since the already limited quality of life is often worsened additionally by the symptoms associated with the dysfunction.

In general, neurogenic bladder dysfunction is thus a disorder of urine storage or urination caused by neurological changes or injuries in the spinal cord in the area of the brain sections relevant to bladder function or in the periphery, wherein normal nervous signal transmission for bladder control is often impaired or disabled. As a result of the underlying neurological damage, the natural function of the bladder is disturbed in terms of its functional states, namely a (urinary) filling phase on the one hand and a (urinary) emptying or micturition phase on the other. Thereby the physiological drain of bladder filling and emptying can even be impaired or prevented on several levels.

As a result of the neurological dysfunction, a neurogenic bladder dysfunction often involves detrusor hyperactivity and/or detrusor-sphincter dyssynergia. This is often accompanied by a disturbance in the interaction or coordination of the anatomical structures relevant to natural bladder function.

The detrusor (musculus detrusor vesicae or “expectorator of the bladder”) is a strong muscle system which surrounds the bladder or forms part of it, wherein the detrusor is a smooth muscle which in a physiological situation plays a major role in micturition or bladder emptying and thus urination. Thereby signals of the parasympathetic nervous system cause a tension of the muscle system, which places the bladder contents under pressure. In addition, the detrusor is influenced by an intrinsic nerve plexus, which lies in the bladder wall and adapts the tone to the filling state of the bladder.

In the pathological situation, neurogenic bladder dysfunction is often associated with detrusor hyperactivity, which in this context is also called neurogenic detrusor overactivity (neurogenic detrusor Qveractivity, NDO). A detrusor hyperactivity, which is associated with a relevant neurological basic disease, is thereby particularly associated with pollakiuria, nocturia and excessive urge symptoms.

Detrusor sphincter dyssynergia (DSD) is also characterized by a disruption of the interaction of the anatomical structures involved in bladder voiding, wherein detrusor hyperactivity is accompanied by spastic dysfunction of the pelvic floor muscles or the external bladder sphincter, which leads to obstruction of the bladder outlet with simultaneous micturition effort. Typical for a detrusor sphincter dyssynergia is thereby a frequently interrupted urinary stream and difficulties in initiating micturition. In addition, pollakiuria and residual urine formation are often present.

In addition to the symptoms mentioned above, a neurogenic bladder dysfunction is often associated with high intravesical pressures, i.e. due to the hyperactivity of the detrusor or detrusor-sphincter dyssynergy, excessive pressure is built up in the bladder. This also poses a risk to the upper urinary tract, i.e. excessive intravesical pressures are often associated with or caused by kidney damage.

In light of the above, there is thus a great need in the prior art to provide therapeutic concepts which lead to a normalization or improvement of bladder function or a reduction of symptoms in the presence of neurogenic bladder dysfunction, both with regard to the problems of urination as well as the high pressures in the bladder. Thereby an essential goal, in addition to improving micturition, is also to reduce the intrinsic pressure, thereby avoiding possible kidney damage or the like.

In this context, the therapy of neurogenic bladder dysfunction thus pursues several goals, especially the protection of the upper urinary tract and renal function, the improvement of the continence situation, the restoration or improvement of the function of the lower urinary tract and, last but not least, the improvement of quality of life.

In addition to conservative measures, such as regular exercise, targeted bladder training or similar, also pharmacotherapeutic measures are used to treat neurogenic bladder dysfunction. For example, on the basis of minimally invasive methods, an injection of botulinum toxin into the bladder wall can be carried out to reduce the activity of the detrusor. However, the administration is relatively complex and can only be carried out by a physician, as the active ingredient has to be injected into the bladder wall in a relatively complex procedure. Furthermore, minimally invasive methods in the form of sacral neuromodulation are also used, in which a pacemaker is implanted in the upper gluteal region, which delivers weak electrical impulses to the sacral nerves via an electrode. However, the field of application is limited with regard to the underlying diseases. Moreover, this is a not insignificant surgical procedure, which is associated with additional risks.

In addition, pharmacological therapies are known in the prior art, in which anticholinergics are also used. In this context, especially the administration of oxybutynin or oxybutynin hydrochloride, trospium chloride, propiverine, tolterodine or the like is considered. Thereby the prior art focuses on the systemic or oral application or administration of these active ingredients. However, systemic administration may be associated with increased systemic side effects such as residual urine formation, constipation, accommodation disorders, dry mouth, tachycardia, cardiac arrhythmia and the like. In addition, the concentration of the active ingredient at the target site, namely the bladder, is sometimes not optimally adjusted.

Furthermore, systemic administration of the active ingredient in the form of anticholinergics is disadvantageous insofar as part of the active ingredient administered is metabolized in the gastrointestinal tract or in the liver during its first passage (so-called first pass metabolism), whereby non-active metabolites are sometimes formed, so that the bioavailability at the target site is lower and the effective quantity at the target site is difficult to adjust or specify. Consequently, patients with systemic dosage forms may not be adequately adjusted.

WO 2012/154779 A1 or US 2012/0289564 A1, which belongs to the same patent family, relates the use of a combination of oxybutynin and a salivary stimulant to treat the overactive bladder, wherein the focus is on oral application.

In addition, anticholinergics can be applied topically, for example with a transdermal patch or the like. Systemic side effects can also occur to a greater extent with this systemic application. In addition, there is also the problem of premature metabolism.

In addition, anticholinergics such as oxybutynin or oxybutynin hydrochloride can be applied or instilled topically into the urogenital area, especially into the bladder, for example by means of a catheter or the like. The local application or instillation of the active ingredient directly at its target site increases the targeted effect or bioavailability and reduces systemic side effects. In addition, the active ingredient is not metabolized prematurely via other organs in this type of administration. In addition, this administration method can also be carried out by the patient himself if necessary.

Oxybutynin or oxybutynin hydrochloride thereby acts especially as a competitive antagonist of acetylcholine on postganionic muscarinic receptors, which leads to a relaxation or antispasmodic effect with regard to the smooth bladder muscles and thus also to a decrease in detrusor hyperactivity. Especially the contraction of the bladder is inhibited, wherein also spasms are loosened. In addition, the administration of oxybutynin or oxybutynin hydrochloride increases bladder volume and reduces contractions, wherein the urge to urinate in neurogenic bladder dysfunction is also reduced or delayed. Furthermore, the use of oxybutynin or oxybutynin hydrochloride can also reduce the intravesical (bladder) pressure, which also leads to a relief of the upper urinary tract. Furthermore, oxybutynin or oxybutynin hydrochloride comprises properties of a local anesthetic. Oxybutynin or oxybutynin hydrochloride thereby belongs especially to the group of anticholinergics.

In the context of the prior art instillation or topical application into the urinary bladder, oxybutynin or oxybutynin hydrochloride is generally used in the form of an aqueous composition or aqueous solution, wherein the active ingredient-comprising composition or solution is applied or administered into the urinary bladder using corresponding application devices.

Thereby the sterility of a composition used for instillation or topical administration into the urinary bladder is of great importance, especially against the background of avoiding possible infections of the urinary bladder, which is particularly susceptible and sometimes already damaged by the clinical picture.

In order to prevent microbial contamination, the active ingredient-comprising compositions can, for example, be manufactured immediately prior to their use or administration, which is costly and also involves a certain risk of contamination of the composition during its manufacture. Furthermore, the active ingredient-comprising compositions as such can be sterilized, for example by microfiltration or similar methods, and afterwards filled into a storage or application device. However, undesirable contamination can also occur in this case, especially with respect to possible germs or the like adhering to the storage or application device. In this context, a separate sterilization of the storage or application device may also be purposeful, but sometimes this also leads to unsatisfactory sterilization results, especially due to the subsequent filling of the composition into the storage or application device. In this context, it is also possible to work under sterile conditions, for example under clean room conditions, but this involves a certain amount of work and is also cost-intensive.

A further problem with sterilization is that the composition comprising the active ingredient to be sterilized may not have the required stability under the necessary or applied sterilization conditions, so that, for example, excessive degradation of the active ingredient may occur, accompanied by the formation of undesirable degradation products, which has a negative effect on both the effectiveness and the application safety of such a composition. Also compositions using oxybutynin or oxybutynin hydrochloride as the active ingredient are somewhat unstable, so that sterilization of the active ingredient may reduce the quality of the composition.

Overall, there is thus a great need in the prior art for corresponding concepts that lead to optimum sterilization of compositions in corresponding holding or application devices with oxybutynin or oxybutynin hydrochloride as the active ingredient, while maintaining the stability of the compositions, in order to obtain ready-to-use products that are highly effective and safe to use for instillation into the urinary bladder (i.e. do not lead to infections if the active ingredient content is high or the amount of degradation products is low). Thereby a high (storage) stability of the composition should also be ensured.

### BRIEF SUMMARY OF THE INVENTION

Against this background, one object of the present invention is to provide an efficient method for producing a sterile composition which comprises oxybutynin or oxybutynin hydrochloride as active ingredient and which is present in a preferably ready-to-use storage or application device, namely a piston syringe, wherein the disadvantages of the prior art described above are at least largely avoided or at least diminished.

Especially, one object of the present invention is to provide a respective method, which leads to a ready-to-use storage or application device, which comprises a corresponding composition with oxybutynin or oxybutynin hydrochloride as active ingredients in sterile form, which even after sterilization has been carried out comprises besides a high sterility also a high or unchanged active ingredient content or at most a low content of corresponding degradation products.

In this respect, in the context of the present invention, especially such a method is to be provided, on the basis of which a corresponding sterile composition with oxybutynin or oxybutynin hydrochloride as active ingredient is provided, which also has a high degree of efficacy and compatibility with a high degree of sterility. Thereby also an improved application safety and an improved (storage) stability and shelf life should be provided. Especially, a ready-to-use product is to be provided on this basis, in which the corresponding composition is provided in a corresponding storage or application device, namely in a corresponding (disposable) syringe or piston syringe.

Especially, within the scope of a further object of the present invention, a storage or application device filled with the active ingredient-comprising composition shall also be provided, namely in the form of a (disposable) syringe, which is accessible to a sterilization process in an improved manner, wherein an effective sterilization shall be ensured while maintaining the stability of the filling material in the form of the active ingredient-comprising composition.

In order to solve the aforementioned object, the present invention thus provides—according to a first aspect of the present invention—a method for producing a sterile oxybutynin-comprising composition in a preferably ready-to-use piston syringe, respective advantageous further configurations and embodiments of this aspect of the invention are the subject-matter of some claims relating to the method.

A further subject-matter of the present invention—according to a second aspect of the present invention—is furthermore the sterile oxybutynin-comprising composition, as it is to be used especially for the prophylactic or therapeutic treatment of neurogenic bladder dysfunctions, as defined in the claims relating to the composition according to the invention, including respective advantageous further configurations and embodiments of this aspect of the invention.

Again a further subject-matter of the present invention—according to a third aspect of the present invention—is also the inventive piston syringe comprising the sterile oxybutynin-comprising composition according to the invention, in accordance with the relevant claims relating to the piston syringe, including respective advantageous further configurations and embodiments of this aspect of the invention.

Another further subject-matter of the present invention—according to a fourth aspect of the present invention—is also the inventive packaging unit comprising the piston syringe according to the invention with the respective composition.

The present invention also relates—according to a fifth aspect of the present invention—to the kit-of-parts according to the respective claim relating to the kit-of-parts.

Again another subject-matter of the present invention—according to a sixth aspect of the present invention—is also the use of a special piston syringe in a method for producing a sterile oxybutynin-comprising composition.

Finally, the subject-matter of the present invention—according to a seventh aspect of the present invention—is the use of vapor sterilization for providing a sterile oxybutynin-comprising composition according to the respective independent claim relating to this use.

It goes without saying that features, embodiments, advantages and the like, which are listed below only with respect to one aspect of the invention for the purpose of avoiding repetition, naturally also correspond to the other aspects of the invention, without this requiring a separate mention.

### BRIEF DESCRIPTION OF THE DRAWINGS

FIG. 1 shows a schematic sectional view of a piston syringe used in the method according to the invention or a piston syringe filled with the sterile oxybutynin-comprising composition as such according to the invention in side view.

### DESCRIPTION

In addition, with regard to the following description of the present invention, the features of the present invention listed in connection with the special configurations, embodiments, advantages, examples or the like of the present invention are also considered disclosed in their combination. Thus, superordinate combinations of individual or several features, which are listed for the respective configurations, embodiments, examples or the like, are also considered disclosed.

In the case of all relative or percentage weight-based data provided below, especially information on quantities or concentrations, it should further be noted that within the scope of the present invention, these must be selected by a person skilled in the art such that they add up to 100% or 100% by weight, respectively, including all components or ingredients, especially as defined below; however, this is self-evident for the person skilled in the art.

Furthermore, it applies that the person skilled in the art—depending on the application or the particular case—may deviate from the concentration, weight, quantity and range specifications listed below without leaving the scope of the present invention.

In addition, it applies that all values or parameters or the like specified in the following can basically be determined with standardized or explicitly specified determination methods and otherwise with determination or measurement methods that are generally used by a person skilled in this field. Unless otherwise provided, the underlying values or parameters are determined under standard conditions (i.e. especially at a temperature of 20° C. and/or at a pressure of 1,013.25 hPa or 1.01325 bar).

For the purpose of illustrating the present invention, the reference signs listed in the FIGURE are also used in the following description of the objects of the invention; the reference signs are thereby purely illustrative and are not accompanied by any limitation of the objects of the invention.

Having stated this, the present invention will be described in more detail hereinafter:

Subject-matter of the present invention—according to a first aspect of the present invention—is thus a method for producing a sterile oxybutynin-comprising composition in a preferably ready-to-use piston syringe, especially a disposable piston syringe, and/or for producing a preferably ready-to-use sterile piston syringe filled with an oxybutynin-comprising composition, especially a disposable piston syringe,

wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber;
wherein the method comprises the following steps:
a) filling the piston syringe with the oxybutynin-comprising composition and/or providing the oxybutynin-comprising composition in the piston syringe,
   wherein the oxybutynin is used in the form of the hydrochloride, the oxybutynin-comprising composition is configured as an aqueous composition and the oxybutynin-comprising composition comprises, in combination and in each case in effective, especially pharmaceutically effective, amounts,
      oxybutynin hydrochloride (oxybutynin-HCl) in a concentration of (1±0.5) mg/ml and
      sodium chloride, calculated as sodium, in a concentration of (3.5±0.5) mg/ml,
   wherein the oxybutynin-comprising composition is adjusted to a pH value in the range of from 3 to 5, especially in the range of from 3.8 to 4.5, and/or wherein the oxybutynin-comprising composition comprises a pH value in the range of from 3 to 5, especially in the range of from 3.8 to 4.5;
      and subsequently
b) vapor sterilization, especially steam sterilization, of the oxybutynin-comprising composition in the piston syringe and/or of the piston syringe filled with the oxybutynin-comprising composition.

In this context, the applicant has found in a completely surprising manner that when using a special sterilization method, namely vapor sterilization, a specially formed oxybutynin-comprising composition can be sterilized in an effective manner using a very specific piston syringe, whereby in this context, within the framework of the method according to the invention, a high stability of the composition is also guaranteed during vapor sterilization. Thus, the oxybutynin-comprising composition sterilized by vapor sterilization according to the inventive method comprises a high active ingredient content even after sterilization, since the active ingredient oxybutynin hydrochloride is only degraded in small amounts during vapor sterilization (which is associated with the formation of only small amounts of degradation products). Thus, according to the invention, the method results in a sterile composition with high application safety and efficiency. Particularly after vapor sterilization, the respective sterilized oxybutynin-comprising composition still has a correspondingly high effectiveness or efficiency.

With regard to the method according to the invention, the corresponding coordination of the components used in the form of the oxybutynin-comprising composition on the one hand and the piston syringe used on the other hand is also of great importance. Thus, with regard to the very specially configured oxybutynin-comprising composition as used in the method according to the invention, the composition comprises a high compatibility with the piston syringe made of cycloolefin-(co)polymer (COP or COC) and the piston plug made of halobutyl rubber, which leads to the high stability of the composition during the specially used vapor sterilization. As a result, the degradation of the active ingredient or the formation of degradation products can be significantly reduced or eliminated.

Thus, with regard to the method according to the invention, the targeted combination of the above-mentioned technical measures, namely the provision of a very special oxybutynin-comprising composition with defined quantities of the components used in this respect and a defined pH value as well as the selection of special materials for the components of the piston syringe used, is of central importance with regard to the vapor sterilization carried out, since in this respect the special selection and adjustment results in a high compatibility during vapor sterilization.

However, the applicant was also able to show that for oxybutynin or oxybutynin hydrochloride in aqueous compositions during thermal sterilization—in contrast to the present invention—there is a high instability or incompatibility in the case of (disposable) syringes based on polypropylene and piston plugs or rubber plugs as well as closing caps made of commonly used rubber materials being used in the course of sterilization. This is because, in contrast to the present invention, sterilization leads to increased degradation of the active ingredient, accompanied by the increased formation of undesirable degradation products, so that the resulting product quality, application safety and effectiveness for this case is impaired overall. In this context, it could also be shown that for compositions comprising oxybutynin hydrochloride as the active ingredient, the degradation product phenylcyclohexylglycolic acid is formed during sterilization using (disposable) syringes based on polypropylene as the syringe material and piston plugs as well as closing caps made of rubber materials usually used for this purpose, so that the product quality is permanently affected. In the context of the present invention, this has now been permanently overcome as presented here.

Within the scope of the present invention, it has thus been possible to overcome the incompatibilities previously shown, and this with a high degree of safety and efficiency with regard to the provision of corresponding sterile compositions.

In this context, it is equally completely surprising that the specific selection of the piston syringe used according to the invention, namely that at least the syringe body is made of a cycloolefin(co)polymer (COP or COC), and according to which the piston plug of the syringe is formed in a special way from a halobutyl rubber (and more preferably from bromobutyl rubber), leads to an increased stability of the composition to be sterilized during vapor sterilization, accompanied by correspondingly reduced amounts of undesirable degradation products and at the same time a high active ingredient content of the sterilized composition. These properties are further improved and guaranteed by the use of a special oxybutynin-comprising composition, which has defined concentrations of oxybutynin hydrochloride and sodium chloride, wherein also a specific pH value is adjusted. According to the invention, on this basis, a high degree of compatibility of the components to be sterilized is thus ensured within the framework of the vapor sterilization process used.

In the context of the present invention, the sterilization of the oxybutynin-comprising composition is based on vapor sterilization in a targeted and purposeful manner, which according to the invention provides excellent sterilization results with simultaneously high product stability.

The method of vapor sterilization thereby belongs to the thermal sterilization methods, wherein the use of (water) vapor also ensures a good energy or heat transfer to the components to be sterilized. Especially when the vapor condenses on the cooler sterilization material, the heat energy or quantity is transferred to the piston syringe or the oxybutynin-comprising composition to be sterilized, which kills or irreversibly inactivates microorganisms or the like. Within the scope of the vapor sterilization used according to the invention, a virtually complete or joint and simultaneous sterilization of the piston syringe used with the oxybutynin-comprising composition is thus ensured, which is a further advantage over sterile filling or similar processes, for example, since this increases the sterilization safety.

In the context of the present invention, the procedure is thus particularly such that a syringe is prefilled with the oxybutynin-comprising composition and made of a special material, as defined hereinabove, is preferably terminally vapor sterilized. This leads to a high sterilization safety and efficiency.

In general, the term (vapor) sterilization as used in the context of the present invention is understood to mean especially the killing or irreversible inactivation of microorganisms, germs, viruses or the like which are present on or in an object to be sterilized, including their resting stages or forms of survival, such as endospores or the like. Since the complete elimination or inactivation of all pathogens on or in an object cannot be guaranteed with complete certainty purely statistically, an object is generally considered sterile if the probability of contamination with pathogens capable of reproduction does not exceed a certain value. In this regard, a value of 1: 10⁶ or 10⁻⁶ at most can be especially given. This means that in less than 1 million units of a sterile item, not more than one unit is non-sterile (e.g. if this unit is contaminated with a colony forming unit (CFU) of a microorganism or pathogen or if the residual pathogen content per unit of sterile item is 10⁻⁶ CFU or less, resulting in a probability of a non-sterile unit of 1: 10⁶). An item can therefore be considered sterile, especially if the theoretical value of not more than one living micro-organism is available in 10⁶ (1 million) sterilized units of the final product. In this context, the Sterility Assurance Level (SAL) is also generally referred to. Reference can also be made to the following explanations.

According to the invention, the material used for the piston syringe or syringe body in the form of cycloolefin-(co)polymer (COP or COC) is a class of technical polymers that can be obtained, for example, by metallocene catalyzed copolymerization of cycloolefins with alk-1-enes. In general, cycloolefin-(co)polymers are amorphous and thus transparent, relative to the visible spectrum of light. Cycloolefin-(co)polymers, as they are used according to the invention, generally comprise high stiffness, strength and hardness as well as low density and high transparency, wherein they are also highly resistant to acids. In addition, the polymers in question also have a high degree of biocompatibility.

As mentioned above, the use of the polymers in question ensures a high degree of compatibility within the framework of the vapor sterilization used according to the invention, which corresponds to improved properties of the sterilized oxybutynin-comprising composition. With regard to the cycloolefin-(co)polymer (COP or COC) used according to the invention, commercially available products can be used (such as TopPAcPolymer Topas COC Grade 6013B-51; Grade EuP 3.2.8).

Furthermore, with regard to the material in the form of halobutyl rubber (and more preferably in the form of bromobutyl rubber) used according to the invention for the piston plug as well as for the closing device (closing cap) of the piston syringe described below, the applicant has found that in this respect, especially in the context of vapor sterilization, there is only a lower chemical reactivity or activity towards oxybutynin hydrochloride. Especially halobutyl rubber, especially bromobutyl rubber, comprises only a lower number of C—C double bonds, so that halobutyl rubber, especially bromobutyl rubber, is less reactive than commonly used rubber materials. In addition, halobutyl rubber, especially bromobutyl rubber as used in the context of the present invention, has further advantages, especially with respect to its high heat stability, improved aging properties and low gas permeability. In this respect, commercially available products can be used (such as Daetwyler V9340, FM257/2 Bromobutyl Compound).

According to the invention, an efficient method for providing a stable and sterile oxybutynin-comprising composition is thus provided on the basis of the targeted combination of all the measures according to the invention.

Within the scope of the present invention, it may especially be provided that the oxybutynin-comprising composition comprises the oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml. Especially, the oxybutynin-comprising composition may contain the oxybutynin hydrochloride in a concentration of 1 mg/ml.

Furthermore, the oxybutynin-comprising composition may comprise the sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml. Especially, the oxybutynin-comprising composition may comprise the sodium chloride, calculated as sodium, in a concentration of 3.5 mg/ml.

With regard to the pH of the oxybutynin-comprising composition according to the invention, it can be adjusted using and/or by means of hydrochloric acid.

According to the invention, it is preferred that the oxybutynin-comprising composition is adjusted to a pH value in the range of 3.8 to 4.5 or has a pH value in the range of 3.8 to 4.5, as previously mentioned. This further improves the stability of the oxybutynin-comprising composition.

With regard to the piston syringe used within the scope of the inventive method, according to the invention it is especially provided that it has a volume, especially a receiving volume for the oxybutynin-comprising composition, in the range of from 4 ml to 25 ml, especially in the range of from 6 ml to 20 ml, preferably in the range of from 8 ml to 15 ml, preferably in the range of from 9 ml to 12 ml, more preferably of about 10 ml. Especially, the piston syringe may comprise a volume of 10 ml. The volume in question thereby especially relates to the corresponding volume of the syringe body or syringe cylinder for the oxybutynin-comprising composition.

According to the invention, the piston syringe can comprise the oxybutynin-comprising composition in an amount in the range of from 4 ml to 25 ml, especially in the range of from 6 ml to 20 ml, preferably in the range of from 8 ml to 15 ml, preferably in the range of from 9 ml to 12 ml, more preferably of about 10 ml. Especially, the piston syringe may comprise the oxybutynin-comprising composition in an amount of 10 ml.

According to the invention, especially a piston syringe with a volume, especially with a receiving volume for the oxybutynin-comprising composition of 10 ml, is used, wherein the piston syringe comprises the oxybutynin-comprising composition in an amount (volume) of 10 ml.

According to the present invention, the piston plug is especially formed from a chlorobutyl rubber or from a bromobutyl rubber, preferably from a bromobutyl rubber.

According to the invention, it may also be provided that the piston syringe, especially the syringe cylinder, comprises a first opening, especially for receiving a syringe piston. In general, the piston plug provided according to the invention is thereby attached or fixed to the end of the syringe piston positioned towards the interior of the syringe body, especially the syringe cylinder.

According to the invention, it may be provided according to a preferred embodiment that the piston syringe comprises a syringe piston, especially wherein the syringe piston is formed from polypropylene or especially wherein the syringe piston is formed from a cycloolefin-(co)polymer (COP or COC). This further improves the properties of the piston syringe during vapor sterilization. In addition, it is advantageous in terms of application and production technology if the syringe body on the one hand and the syringe piston on the other hand of the piston syringe used according to the invention are formed from the same material in the form of cycloolefin-(co)polymer (COP or COC).

Furthermore, according to the invention, it is particularly intended that the piston syringe comprises a second opening, especially a discharge opening, preferably for discharging and/or applying the oxybutynin-comprising composition.

Furthermore, according to the invention, the piston syringe may also comprise a closing element, especially a closing cap, preferably for closing the second opening. At the same time, according to the present invention, it is especially intended that the closing element is formed from a halobutyl rubber, especially from a chlorobutyl rubber or from a bromobutyl rubber, preferably from a bromobutyl rubber.

According to the invention, it is thus preferably provided that the piston syringe comprises a closing element, especially a closing cap, made of a bromobutyl rubber. For this purpose, a bromobutyl rubber similar to that previously mentioned or used for the piston plug can be used. The configuration of the closing element in the form of a bromobutyl rubber further improves the compatibility of the overall components used and the stability of the oxybutynin-comprising composition sterilized according to the invention during sterilization, especially with regard to the reduction of the formation of degradation products of oxybutynin hydrochloride. In this respect, reference is also made to the explanations regarding the piston plug used according to the invention. The closing element can be made of commercially available materials (such as Datwyler FM252-2 Bromobutyl Grade Eu.P. 3.2.9).

According to the invention, it is especially intended that the piston syringe with the oxybutynin-comprising composition inserted therein is closed with the closing element at the second opening of the piston syringe during vapor sterilization.

In this respect, according to the invention, it is generally intended that, within the scope of the vapor sterilization, preferably the complete unit as such, which comprises the piston syringe filled with the oxybutynin-comprising composition with the relevant syringe body, the piston or syringe plug, the closing element and, if applicable, the syringe piston, is sterilized.

According to the invention, vapor sterilization is generally carried out under the application of temperature (heating). In this respect, according to the invention, it has been shown to be advantageous if vapor sterilization is carried out at temperatures of at least 105° C., especially of at least 110° C., preferably of at least 115° C., preferably of at least 120° C. Especially, vapor sterilization should be carried out at temperatures of at most 160° C., especially of at most 150° C., preferably of at most 140° C., preferably of at most 130° C.

In general, according to the invention, vapor sterilization can be carried out at temperatures in the range of from 105° C. to 160° C., especially in the range of from 110° C. to 150° C., preferably in the range of from 115° C. to 140° C., preferably in the range of from 20° C. to 130° C., more preferably of about 121° C. Especially, vapor sterilization can be carried out at 121° C. according to the present invention.

Effective sterilization can be carried out at the above-mentioned temperatures, wherein appropriate protection of the material is also guaranteed. The temperatures in question thereby refer especially to the coldest point or position in a sterilization room of a sterilization device used for this purpose.

According to the invention, it may be provided that vapor sterilization is carried out for a duration of at least 1 min, especially of at least 2 min, preferably of at least 3 min, preferably of at least 5 min, more preferably of at least 8 min, most preferably of at least 10 min, even more preferably of at least 15 min, yet even more preferably of at least 17 min.

In addition, according to the invention, it may be provided that vapor sterilization is carried out for a duration of at most 500 min, especially of at most 400 min, preferably of at most 200 min, preferably of at most 100 min, more preferably of at most 60 min, most preferably of at most 45 min, even more preferably of at most 30 min, yet even more preferably of at most 25 min.

According to the invention, vapor sterilization can generally be carried out for a duration in the range of from 1 min to 500 min, especially in the range of from 2 min to 400 min, preferably in the range of from 3 min to 200 min, preferably in the range of from 5 min to 100 min, more preferably in the range of from 8 min to 60 min, most preferably in the range of from 10 min to 45 min, even more preferably in the range of from 15 min to 30 min, yet even more preferably in the range of from 17 min to 25 min, yet again more preferably of about 20 min. According to the invention, effective sterilization can thus be achieved after relatively short sterilization times.

According to the invention, vapor sterilization can also be carried out under pressurization or under elevated pressure. This can further increase the sterilization efficiency.

In the context of the present invention, the term “pressurization” or “elevated pressure” is to be understood especially as the pressure of the gas phase present during vapor sterilization, which is higher than the pressure under standard conditions (1.013 bar at 25° C.) or than the pressure of the surrounding atmosphere (generally about 1 bar). Increased pressure can therefore be characterized or detected by a positive amount of relative pressure. The “relative pressure” of a system is generally understood to be especially the pressure difference between the absolute pressure of the gas phase of the system in question (for example, the pressure inside an autoclave) and the absolute pressure of the surrounding atmosphere. As far as the term “absolute pressure” used according to the invention is concerned, it is particularly understood to mean the pressure opposite to zero pressure in empty space (vacuum).

According to the invention, vapor sterilization can be carried out especially at a relative pressure in the range of from 0.1 bar to 10 bar, especially in the range of from 0.5 bar to 5 bar, preferably in the range of from 1 bar to 3.5 bar, preferably in the range of from 1.5 bar to 3 bar. Especially, vapor sterilization can be carried out at an absolute pressure in the range of from 1.1 bar to 10 bar, especially in the range of from 1.5 bar to 6 bar, preferably in the range of from 2 bar to 4.5 bar. The pressure range in question also ensures sufficient condensation of the vapor on the material to be sterilized, accompanied by good energy and heat transfer.

In general, the present invention may provide for vapor sterilization to be carried out in the presence of an atmosphere comprising especially pure steam and/or as a saturated steam process. However, according to the invention, it may also be provided that the vapor sterilization is carried out in the presence of a steam/gas mixture, especially a mixture of steam with inert gas, especially nitrogen, and/or oxygen, preferably a steam/air mixture

In this context, saturated steam is to be understood especially as a saturated atmosphere of pure steam (water vapor) (wherein pure steam, especially according to the European standard EN 285, may contain up to 3.5% by volume of non-condensable gases, such as oxygen, nitrogen, etc.), while an atmosphere comprising pure steam may also be, for example, an atmosphere of superheated vapor.

According to the invention, vapor sterilization should be carried out in a preferably closed sterilization device, especially in a gas-tightly closed pressure vessel, preferably in an autoclave apparatus (autoclave). The corresponding apparatus or devices are well known to the person skilled in the art, so that no further explanations are necessary.

Within the scope of the present invention, it may especially be intended that, during vapor sterilization, steam is generated from distilled or demineralized water. Especially, during vapor sterilization, compressed air can be generated from sterile air, especially wherein the air is cleaned or sterilized by passing through filters. Especially, in this respect particle filters, such as HEPA filters or similar, can be used. This especially prevents that no additional microorganisms are introduced into the sterilization equipment.

According to the invention, vapor sterilization can be carried out under overkill conditions and/or at about 121° C. and/or for a duration of at least 15 min, especially of at least 20 min, preferably of about 20 min. Especially, vapor sterilization can be carried out at 121° C. and for a duration of 20 minutes. This gives particularly good sterilization results. The overkill conditions stated according to the invention are thereby especially based on a further development of the reference method for vapor sterilization as stated in Pharmacopoea Europaea (Ph. Eur.), Chapter 5.1.1 (07/2017:50101).

According to the invention, it has also proven to be advantageous if vapor sterilization is carried out according to Pharmacopoea Europaea (Ph. Eur.), Chapter 5.1.1 (07/2017:50101). In this respect, according to the invention, especially overkill conditions are used. In this respect, especially the process or vapor sterilization can be carried out at a temperature of about 121° C. or for a duration of at least 15 min, especially at least 20 min, preferably about 20 min. In this respect, reference can also be made to the corresponding explanations in Pharmacopoea Europaea (Ph. Eur.), Chapter 5.1.1 (07/2017:50101), wherein the relevant disclosure content is hereby fully included by reference.

According to the invention, it is also possible to proceed in such a way that vapor sterilization is carried out multi-cyclically. In this respect, at least the final and/or terminal cycle can be carried out under at least one of the previously defined (vapor sterilization) conditions or parameters. In this context, at least the final and/or terminal cycle and/or terminal vapor sterilization can be carried out under overkill conditions and/or at about 121° C. and/or for a duration of at least 15 min, especially of at least 20 min, preferably of about 20 min.

In accordance with the invention, it may in principle be intended that the piston syringe filled with the oxybutynin-comprising composition has been inserted, especially prior to the performance of the vapor sterilization, into a packaging, especially a stand-by packaging, which is closed and/or sealed, especially in a water-tight and/or germ-proof manner. In this context, according to the invention, it is especially intended that at least part of the packaging is configured to be at least steam-permeable, especially steam- and/or gas-permeable. This also ensures good sterilization of the inserted or packaged piston syringe with the oxybutynin-comprising composition. In accordance with the invention, it thereby behaves especially in such a way that the optionally present packaging is also sterilized when the vapor sterilization is performed.

In accordance with the invention, it may also be provided that a plurality of piston syringes filled with the oxybutynin-comprising composition is sterilized simultaneously. In this context, it may especially be intended that, preferably during the performance of the vapor sterilization, several piston syringes are brought together on a (syringe) carrier

Overall, excellent sterilization results are obtained within the scope of the inventive method. In this context, according to the invention, it is especially intended that the sterile oxybutynin-comprising composition obtained after vapor sterilization and/or the sterile piston syringe filled with the oxybutynin-comprising composition obtained after vapor sterilization has a SAL (Sterility Assurance Level) value of at least 10⁻⁵, especially of at least 10⁻⁶, preferably of at least 10⁻⁷.

Especially, according to the invention, it may be provided that the vapor sterilized or sterile oxybutynin-comprising composition or the piston syringe obtained after vapor sterilization and filled with the oxybutynin-comprising composition is sterile according to DIN EN 566-1.

For further details on the vapor sterilization on which the method according to the invention is based, reference can be made especially to DE 10 2017 104 931 A1 and WO 2018/145799 A1, which belong to the same patent family, wherein the relevant disclosure is hereby fully disclosed by reference. In addition, reference can also be made to DE 10 2011 105 840 A1 and WO 2012/136313 A2, which belong to the same patent family, and to US 2014/093422 A1, which respective disclosure is hereby also fully included by reference.

As mentioned above, a sterile oxybutynin-comprising composition with high stability is provided within the scope of the inventive method. Especially, the sterile oxybutynin-comprising composition has a low content of impurities or degradation products.

In this context, the sterile oxybutynin-comprising composition obtained after vapor sterilization has a content of degradation product(s) of oxybutynin, especially a content of phenylcyclohexylglycolic acid, of at most 10% by weight, especially of at most 7.5% by weight, preferably of at most 5% by weight, preferably of at most 2% by weight, more preferably of at most 1% by weight, based on oxybutynin hydrochloride.

According to the present aspect, the present invention also relates to a method for producing a sterile oxybutynin-comprising composition in a preferably ready-to-use piston syringe, especially a disposable piston syringe, and/or for producing a preferably ready-to-use, sterile piston syringe filled with an oxybutynin-comprising composition, especially a disposable piston syringe, especially a method as defined hereinabove,

wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber;
wherein the method comprises the following steps:
a) filling the piston syringe with the oxybutynin-comprising composition and/or providing the oxybutynin-comprising composition in the piston syringe,
   wherein the oxybutynin is used in the form of the hydrochloride, the oxybutynin-comprising composition is configured as an aqueous composition and the oxybutynin-comprising composition comprises, in combination and in each case in effective, especially pharmaceutically effective, amounts,
      oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml, and
      sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5;
      and subsequently
b) vapor sterilization, especially steam sterilization, of the oxybutynin-comprising composition in the piston syringe and/or of the piston syringe filled with the oxybutynin-comprising composition.

Furthermore, according to this aspect of the present invention, the present invention also relates to a method for producing a sterile oxybutynin-comprising composition in a preferably ready-to-use piston syringe, especially a disposable piston syringe, and/or for producing a preferably ready-to-use sterile piston syringe filled with an oxybutynin-comprising composition, especially a disposable piston syringe, especially a method according to any of the preceding claims,

wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber;
wherein the method comprises the following steps:
a) filling the piston syringe with the oxybutynin-comprising composition and/or providing the oxybutynin-comprising composition in the piston syringe,
   wherein the oxybutynin is used in the form of the hydrochloride, the oxybutynin-comprising composition is configured as an aqueous composition and the oxybutynin-comprising composition comprises, in combination and in each case in effective, especially pharmaceutically effective, amounts,
      oxybutynin hydrochloride (oxybutynin-HCl) in a concentration of about 1 mg/ml and
      sodium chloride, calculated as sodium, in a concentration of about 3.5 mg/ml,
   wherein the oxybutynin-comprising composition is adjusted to a pH value in the range of from 3.8 to 4.5 and/or wherein the oxybutynin-comprising composition has a pH value in the range of from 3.8 to 4.5;
      and subsequently
b) vapor sterilization, especially steam sterilization, of the oxybutynin-comprising composition in the piston syringe and/or of the piston syringe filled with the oxybutynin-comprising composition.

Furthermore, according to this aspect, the present invention also relates to a method for producing a sterile oxybutynin-comprising composition in a preferably ready-to-use piston syringe, especially a disposable piston syringe, and/or for producing a preferably ready-to-use sterile piston syringe filled with an oxybutynin-comprising composition, especially a disposable piston syringe, especially a method as defined hereinabove,

wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber;
wherein the method comprises the following steps:
a) filling the piston syringe with the oxybutynin-comprising composition and/or providing the oxybutynin-comprising composition in the piston syringe,
   wherein the oxybutynin is used in the form of the hydrochloride, the oxybutynin-comprising composition is configured as an aqueous composition and the oxybutynin-comprising composition comprises, in combination and in each case in effective, especially pharmaceutically effective, amounts,
      oxybutynin hydrochloride (oxybutynin-HCl) in a concentration of about 1 mg/ml and
      sodium chloride, calculated as sodium, in a concentration of about 3.5 mg/ml,
   wherein the oxybutynin-comprising composition is adjusted to a pH value in the range of from 3.8 to 4.5 and/or wherein the oxybutynin-comprising composition has a pH value in the range of from 3.8 to 4.5;
      and subsequently
b) vapor sterilization, especially steam sterilization, of the oxybutynin-comprising composition in the piston syringe and/or of the piston syringe filled with the oxybutynin-comprising composition,
   wherein the vapor sterilization is carried out under overkill conditions and/or at about 121° C. and/or for a duration of at least 15 min, especially of at least 20 min, preferably of about 20 min, and/or
      wherein the vapor sterilization is carried out according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

With respect to the configuration of the method according to the invention in accordance with the present aspect, reference can also be made to the following statements on the further aspects according to the invention, which apply accordingly.

Furthermore, the subject-matter of the present invention—according to a further aspect of the present invention—is also the sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,

wherein the oxybutynin-comprising composition is obtainable by the method according to the present invention; and/or
wherein the oxybutynin-comprising composition has been vapor sterilized, especially steam-sterilized, according to the method according to the present invention.

According to this aspect, the present invention also relates to the sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy, especially the composition defined hereinabove,

wherein the oxybutynin-comprising composition is present in a preferably ready-to-use piston syringe, especially a disposable piston syringe,
wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber;
wherein the oxybutynin-comprising composition comprises the oxybutynin in the form of the hydrochloride, the oxybutynin-comprising composition is configured as an aqueous composition and the oxybutynin-comprising composition comprises, in combination and in each case in effective, especially pharmaceutically effective, amounts,
oxybutynin hydrochloride (oxybutynin-HCl) in a concentration of (1±0.5) mg/ml and
sodium chloride, calculated as sodium, in a concentration of (3.5±0.5) mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the oxybutynin-comprising composition comprised in the piston syringe, including the piston syringe, is vapor sterilized, especially steam-sterilized.

In this context, the oxybutynin-comprising composition may comprise the oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml.

Furthermore, the oxybutynin-comprising composition may comprise the sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml.

According to the invention, it is especially preferred that the piston plug is formed from a chlorobutyl rubber or from a bromobutyl rubber, preferably from a bromobutyl rubber.

In general, the oxybutynin-comprising composition may, according to the invention, be vapor sterilized under at least one or one of the (vapor sterilization) conditions or parameters previously defined for the method according to the invention.

According to the invention, it also behaves especially such that the oxybutynin-comprising composition is vapor sterilized under overkill conditions and/or at about 121° C. and/or for a duration of at least 15 min, especially of at least 20 min, preferably of about 20 min.

Especially, according to the invention, it may be provided that the oxybutynin-comprising is vapor sterilized according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

Especially, the sterile oxybutynin-comprising composition obtained after vapor sterilization and/or the sterile piston syringe filled with the oxybutynin-comprising composition obtained after vapor sterilization may have a SAL (Sterility Assurance) value of at least 10⁻⁵, especially of at least 10⁻⁶, preferably of at least 10⁻⁷.

As mentioned above, the sterile oxybutynin-comprising composition has a high stability. Especially, the sterile oxybutynin-comprising composition obtained after vapor sterilization may have a content of degradation product(s) of oxybutynin, especially a content of phenylcyclohexylglycolic acid, of at most 10% by weight, especially of at most 7.5% by weight, preferably of at most 5% by weight, preferably of at most 2% by weight, more preferably of at most 1% by weight, based on oxybutynin hydrochloride.

As mentioned above, the composition according to the invention can be used especially for the prophylactic or therapeutic treatment of neurogenic bladder dysfunction. Thus, the present invention also concerns the use of the sterile oxybutynin-comprising composition (for producing a drug) for the treatment of neurogenic bladder dysfunction. The present invention particularly concerns the sterile oxybutynin-comprising composition according to the invention for use in the treatment of neurogenic bladder dysfunction.

According to the invention, it is generally the case that the neurogenic bladder dysfunctions are associated with and/or characterized by detrusor overactivity and/or detrusor-sphincter dyssynergia, especially overactive detrusor.

Especially, the neurogenic bladder dysfunctions may be associated with or caused by spinal cord injury, especially paraplegia, spina bifida, diabetes, multiple sclerosis, stroke or Parkinson's disease.

According to the present aspect of the invention, the present invention also relates to a sterile oxybutynin-comprising composition, especially for use in the prophylactic and/or therapeutic treatment of neurogenic bladder dysfunction, especially neurogenic voiding disorders, preferably neurogenic bladder dysfunction associated with detrusor hyperactivity and/or detrusor sphincter dyssynergy, especially a composition as previously defined,

wherein the oxybutynin-comprising composition is present in a preferably ready-to-use piston syringe, especially a disposable piston syringe,
wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or bromobutyl rubber, preferably from a bromobutyl rubber;
wherein the oxybutynin-comprising composition comprises the oxybutynin in the form of the hydrochloride, the oxybutynin-comprising composition is configured as an aqueous composition and the oxybutynin-comprising composition comprises, in combination and in each case in effective, especially pharmaceutically effective, amounts,
oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml, and
sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the oxybutynin-comprising composition comprised in the piston syringe, including the piston syringe, is vapor sterilized, especially steam-sterilized.

According to the present aspect, the present invention also relates to the sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy, especially a composition as previously defined,

wherein the oxybutynin-comprising composition is present in a preferably ready-to-use piston syringe, especially a disposable piston syringe,
wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber;
wherein the oxybutynin-comprising composition comprises the oxybutynin in the form of the hydrochloride, the oxybutynin-comprising composition is configured as an aqueous composition and the oxybutynin-comprising composition comprises, in combination and in each case in effective, especially pharmaceutically effective, amounts,
oxybutynin hydrochloride (oxybutynin-HCl) in a concentration of about 1 mg/ml and
sodium chloride, calculated as sodium, in a concentration of about 3.5 mg/ml,
   wherein the oxybutynin-comprising composition is adjusted to a pH value in the range of from 3.8 to 4.5, and/or wherein the oxybutynin-comprising composition has a pH value in the range of from 3.8 to 4.5; and
   wherein the oxybutynin-comprising composition comprised in the piston syringe, including the piston syringe, is vapor sterilized, especially steam-sterilized.

According to the present aspect, the present invention further relates to the sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy, especially a composition as previously defined,

wherein the oxybutynin-comprising composition is present in a preferably ready-to-use piston syringe, especially a disposable piston syringe,
wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber;
wherein the oxybutynin-comprising composition comprises the oxybutynin in the form of the hydrochloride, the oxybutynin-comprising composition is configured as an aqueous composition and the oxybutynin-comprising composition comprises, in combination and in each case in effective, especially pharmaceutically effective, amounts,
oxybutynin hydrochloride (oxybutynin-HCl) in a concentration of about 1 mg/ml and
sodium chloride, calculated as sodium, in a concentration of about 3.5 mg/ml,
   wherein the oxybutynin-comprising composition is adjusted to a pH value in the range of from 3.8 to 4.5, and/or wherein the oxybutynin-comprising composition has a pH value in the range of from 3.8 to 4.5; and
   wherein the oxybutynin-comprising composition comprised in the piston syringe, including the piston syringe, is vapor sterilized, especially steam-sterilized,
   wherein the vapor sterilization is carried out under overkill conditions and/or at about 121° C. and/or for a duration of at least 15 min, especially of at least 20 min, preferably of about 20 min, and/or
   wherein the vapor sterilization is carried out according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

As previously mentioned, the present invention also relates to the sterile oxybutynin-comprising composition according to the invention for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy. In this context, the neurogenic bladder dysfunction disorders may be associated with or characterized by detrusor overactivity and/or detrusor sphincter dyssynergy, especially overactive detrusor. In this context, neurogenic bladder dysfunction may also be associated with or caused by spinal cord injury, especially paraplegia, spina bifida, diabetes, multiple sclerosis, stroke or Parkinson's disease.

Especially, the composition according to the invention may be intended or prepared for instillation and/or for preferred topical application into the urogenital region, especially into the bladder.

Furthermore, the oxybutynin-comprising composition according to the invention may be intended or prepared for administration and/or instillation and/or for preferably topical application into the urogenital tract, especially into the bladder.

Especially, the oxybutynin-comprising composition can be administered by instillation and/or by topical application into the urogenital tract, especially into the bladder.

With regard to further configurations of the composition according to the invention, reference can also be made to the explanations on the further aspects according to the invention, which apply according in the present case.

In addition, the subject-matter of the present invention—according to a further aspect of the present invention—is furthermore the piston syringe according to the present invention, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber,

wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy, especially as defined above,
wherein the piston syringe is obtainable by a method as defined above; and/or
wherein the oxybutynin-comprising composition comprised in the piston syringe, including the piston syringe, has been vapor sterilized, especially steam-sterilized, by a method as described above.

According to this aspect, the present invention particularly relates to the piston syringe according to the present invention, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially a piston syringe as defined above,

wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergia,
wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
oxybutynin hydrochloride (oxybutynin-HCl) in a concentration of (1±0.5) mg/ml and
sodium chloride, calculated as sodium, in a concentration of (3.5±0.5) mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the oxybutynin-comprising composition comprised in the piston syringe, including the piston syringe, is vapor sterilized, especially steam-sterilized.

In general, the oxybutynin-comprising composition may comprise the oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml.

In addition, the oxybutynin-comprising composition may comprise the sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml. Especially, the oxybutynin-comprising composition may contain the sodium chloride, calculated as sodium, in a concentration of 3.5 mg.

According to the invention, the piston syringe may have a volume, especially a receiving volume for the oxybutynin-comprising composition, in the range of from 4 ml to 25 ml, especially in the range of from 6 ml to 20 ml, preferably in the range of from 8 ml to 15 ml, preferably in the range of from 9 ml to 12 ml, more preferably of about 10 ml.

Similarly, the piston syringe may comprise the oxybutynin-comprising composition in an amount (volume) in the range of from 4 ml to 25 ml, especially in the range of from 6 ml to 20 ml, preferably in the range of from 8 ml to 15 ml, preferably in the range of from 9 ml to 12 ml, more preferably of about 10 ml. Especially, the piston syringe may comprise the oxybutynin-comprising composition in a volume of 10 ml.

According to the invention, it is preferred that the piston plug is formed from a chlorobutyl rubber or from a bromobutyl rubber, preferably from a bromobutyl rubber.

The piston syringe according to the invention, especially the syringe cylinder, can generally comprise a first opening, especially for receiving a syringe piston.

According to the invention, it may especially be provided that the piston syringe comprises a syringe piston. In this context, according to the present invention, it is preferred that the syringe piston is formed from polypropylene or especially wherein the syringe piston is formed from a cycloolefin-(co)polymer (COP or COC).

In general, the piston syringe may comprise a second opening, especially a discharge opening, preferably for discharging and/or applying the oxybutynin-comprising composition. In this respect, the piston syringe may comprise a closing element, especially a closing cap, preferably for closing the second opening. According to the invention, it is thereby particularly preferred that the closing element is formed from a halobutyl rubber, especially from a chlorobutyl rubber or from a bromobutyl rubber, preferably from a bromobutyl rubber.

According to the invention, vapor sterilization can be carried out under at least one or one of the (vapor sterilization) conditions or parameters previously defined for the method according to the invention.

With regard to the piston syringe according to the invention, vapor sterilization can be carried out especially under overkill conditions and/or at about 121° C. and/or for a duration of at least 15 min, especially of at least 20 min, preferably of about 20 min.

Especially, vapor sterilization can be carried out according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

Furthermore, as far as the piston syringe according to the invention is concerned, the sterile piston syringe filled with the sterile oxybutynin-comprising composition obtained after vapor sterilization and/or the oxybutynin-comprising composition obtained after vapor sterilization can comprise a SAL value (Sterility Assurance) of at least 10⁻⁵, especially of at least 10⁻⁶, preferably of at least 10⁻⁷.

The sterile piston syringe can be used especially for instillation and/or for the preferred topical application of the oxybutynin-comprising composition into the urogenital region, especially into the bladder.

According to the present aspect, the present invention also relates to a piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber, especially a piston syringe as defined above,

wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml, and
sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and wherein the oxybutynin-comprising composition comprised in the piston syringe, including the piston syringe, is vapor sterilized, especially steam-sterilized.

According to the present aspect, the present invention equally relates to a piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber, especially a piston syringe as defined above, wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,

wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml, and
sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the oxybutynin-comprising composition comprised in the piston syringe, including the piston syringe, is vapor sterilized, especially steam-sterilized.

The present aspect equally comprises a piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber, especially a piston syringe as defined above,

wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
oxybutynin hydrochloride (oxybutynin-HCl) in a concentration of about 1 mg/ml and
sodium chloride, calculated as sodium, in a concentration of about 3.5 mg/ml,
   wherein the oxybutynin-comprising composition is adjusted to a pH value in the range of from 3.8 to 4.5 and/or wherein the oxybutynin-comprising composition has a pH value in the range of from 3.8 to 4.5; and
   wherein the oxybutynin-comprising composition comprised in the piston syringe, including the piston syringe, is vapor sterilized, especially steam-sterilized.

According to the present aspect, the present invention also relates to a piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber, especially a piston syringe as defined above,

wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
oxybutynin hydrochloride (oxybutynin-HCl) in a concentration of about 1 mg/ml and
sodium chloride, calculated as sodium, in a concentration of about 3.5 mg/ml,
   wherein the oxybutynin-comprising composition is adjusted to a pH value in the range of from 3.8 to 4.5 and/or wherein the oxybutynin-comprising composition has a pH value in the range of from 3.8 to 4.5; and
   wherein the oxybutynin-comprising composition comprised in the piston syringe, including the piston syringe, is vapor sterilized, especially steam-sterilized,
   wherein the vapor sterilization is carried out under overkill conditions and/or at about 121° C. and/or for a duration of at least 15 min, especially of at least 20 min, preferably of about 20 min, and/or
   wherein the vapor sterilization is carried out according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

According to the present aspect, the present invention also relates to a piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber, especially a piston syringe a defined above,

wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
oxybutynin hydrochloride (oxybutynin-HCl) in a concentration of about 1 mg/ml and
sodium chloride, calculated as sodium, in a concentration of about 3.5 mg/ml,
   wherein the oxybutynin-comprising composition is adjusted to a pH value in the range of from 3.8 to 4.5 and/or wherein the oxybutynin-comprising composition has a pH in the range of from 3.8 to 4.5; and
   wherein the piston syringe comprises a volume, especially a receiving volume for the oxybutynin-comprising composition, of about 10 ml, and/or wherein the piston syringe comprises the oxybutynin-comprising composition in an amount of about 10 ml,
   wherein the oxybutynin-comprising composition comprised in the piston syringe, including the piston syringe, is vapor sterilized, especially steam-sterilized.

With regard to further configurations of the piston syringe according to the invention, reference can also be made to the explanations on the other aspects according to the invention, which apply correspondingly to the present case.

Furthermore, the present invention—according to another aspect of the present invention—also relates to the packaging unit according to the invention, comprising at least one packaging and at least one piston syringe, especially disposable piston syringe, as defined above, wherein the piston syringe is inserted into the packaging and/or is present in the packaging.

In this context, it may especially behave according to the invention in such a way that the packaging unit according to the invention or the respective packaging with the respective piston syringe and the oxybutynin-comprising composition has been vapor sterilized according to the method according to the invention, as defined above.

In addition, the packaging unit may generally be configured as a ready-to-use package. Especially, the packaging unit can be closed or sealed water- and/or germ-proof according to the invention. Furthermore, at least part of the packaging may be configured to be at least steam-permeable, especially steam- and/or gas-permeable.

For further explanations or configurations of the packaging unit according to the invention, reference can also be made to the further explanations on the further aspects of the present invention, which apply accordingly.

Again a further subject-matter of the present invention—according to a further aspect of the present invention—is furthermore the kit according to the invention, especially an application and/or instillation system, comprising (i) at least one oxybutynin-comprising composition, as defined above, present in a piston syringe, especially disposable piston syringe, especially as defined above; (ii) (a) at least one application and/or instillation device connectable to the piston syringe, especially in the form of an instillation tube or the like, preferably in the form of a (urinary bladder) catheter, and/or (b) at least one connection device, especially an adapter, connectable to the piston syringe for connecting to an application and/or instillation device, especially in the form of an instillation tube or the like, preferably in the form of a (urinary bladder) catheter; and optionally (iii) at least one application and/or instillation manual.

According to this aspect, the present invention also relates to the kit according to the invention, especially an application and/or instillation system, comprising (i) at least one piston syringe, especially a disposable piston syringe, as defined above, and/or at least one packaging unit, as defined in above; (ii) (a) at least one application and/or instillation device connectable to the piston syringe, especially in the form of an instillation tube or the like, preferably in the form of a (urinary bladder) catheter, and/or (b) at least one connecting device, especially an adapter, connectable to the piston syringe for connecting to an application and/or instillation device, especially in the form of an instillation tube or the like, preferably in the form of a (urinary bladder) catheter; and optionally (iii) at least one application and/or instillation manual.

With regard to further configurations in this respect, reference can also be made to the explanations on the other aspects according to the invention, which apply accordingly.

Another subject-matter of the present invention—according to another aspect of the present invention—is the use of a piston syringe, especially a disposable piston syringe, in a method for producing a sterile oxybutynin-comprising composition, especially as defined above,

wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber,
wherein the piston syringe is filled with the sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergia,
wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
oxybutynin hydrochloride (oxybutynin-HCl) in a concentration of (1±0.5) mg/ml and
sodium chloride, calculated as sodium, in a concentration of (3.5±0.5) mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and wherein the oxybutynin-comprising composition comprised in the piston syringe, including the piston syringe, is vapor sterilized, especially steam-sterilized.

In this respect, the explanations on the other aspects according to the invention may also apply, which apply accordingly in the present case.

Finally, another subject-matter of the present invention—according to a further aspect of the present invention—is also the use of vapor sterilization according to the present invention, especially steam sterilization, in a method for producing a sterile oxybutynin-comprising composition in a preferably ready-to-use piston syringe, especially disposable piston syringe, and/or for producing a preferably ready-to-use, sterile piston syringe filled with an oxybutynin-comprising composition, especially disposable piston syringe, especially as defined above,

wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber;
wherein in the oxybutynin-comprising composition the oxybutynin is used in the form of the hydrochloride, the oxybutynin-comprising composition is configured as an aqueous composition and the oxybutynin-comprising composition comprises, in combination and in each case in effective, especially pharmaceutically effective, amounts,
oxybutynin hydrochloride (oxybutynin-HCl) in a concentration of (1±0.5) mg/ml and
sodium chloride, calculated as sodium, in a concentration of (3.5±0.5) mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the piston syringe filled with the oxybutynin-comprising composition and/or the oxybutynin-comprising composition provided in the piston syringe is vapor sterilized, especially steam-sterilized.

In this respect, for further explanations regarding the use according to the invention according to this aspect, reference can also be made to the explanations regarding the other aspects according to the invention, which apply accordingly in the present case.

Hereinafter, the present invention will also be described on the basis of a FIGURE elucidation using a preferred embodiment or configuration. In connection with the explanation of this preferred embodiment or configuration of the present invention, which however is in no way restrictive with respect to the present invention, further advantages, properties, aspects and features of the present invention are also shown.

The FIGURE representation according to

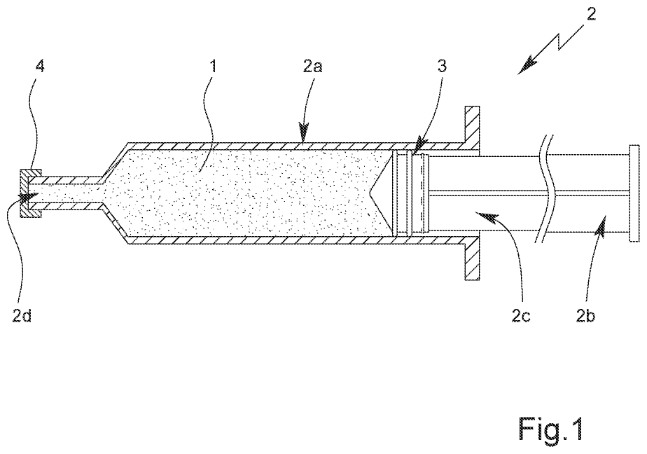

FIG. 1 shows a schematic sectional view of a piston syringe used in the method according to the invention or a piston syringe filled with the sterile oxybutynin-comprising composition as such according to the invention in side view.

Especially, FIG. 1 also shows the sterile oxybutynin-comprising composition 1 according to the invention, with which the piston syringe 2 is filled, wherein the piston syringe 2 is especially configured as a disposable piston syringe and wherein the piston syringe 2 comprises a syringe body 2a, especially a syringe cylinder, on the one hand and a syringe piston 2b with a piston plug 3 on the other hand. In this context, the syringe body 2a is formed from a cycloolefin-(co)polymer (COP or COC), and the piston plug 3 is formed from a halobutyl rubber, especially from a chlorobutyl rubber or from a bromobutyl rubber, preferably from a bromobutyl rubber. FIG. 1 shows that the piston syringe 2 is filled with the sterile oxybutynin-comprising composition 1 according to the present invention. FIG. 1 further illustrates that the piston syringe 2 according to the invention comprises a first opening 2c for receiving the syringe piston 2b with the piston plug 3 and that the piston syringe 2 according to the invention also comprises a second opening 2d, especially opposite of the first opening 2c, especially a discharge opening. The second opening 2d is closed with a closing element 4 in the form of a closing cap, wherein the closing element or the closing cap is formed from a halobutyl rubber, especially from a chlorobutyl rubber or from a bromobutyl rubber, preferably from a bromobutyl rubber. FIG. 1 thus shows the closed state of the piston syringe with the oxybutynin-comprising composition therein according to the invention. Within the scope of the method according to the invention, this unit consisting of a sealed piston syringe with the oxybutynin-comprising composition comprised can be vapor sterilized, so that in this way a corresponding sterile oxybutynin-comprising composition or a sterile piston syringe filled with the composition according to the invention is obtained.

Further configurations, modifications and variations as well as advantages of the present invention are easily recognizable and realizable for the person skilled in the art reading the description without leaving the scope of the present invention.

The following examples are merely intended to illustrate the present invention, but without limiting the present invention to this.

### Exemplary Embodiments

The explanations described below show the improved properties of the sterile oxybutynin-comprising composition provided on the basis of the method according to the invention, namely with respect to the improved stability of the composition during sterilization with the reduction of degradation products of oxybutynin hydrochloride.

Particularly in the case of terminal vapor sterilization of a composition for instillation into the bladder, which comprises the active ingredient oxybutynin hydrochloride in a concentration of 1 mg/ml at a pH value of the composition of 4.0, and which—in contrast to the present invention—is prefilled in polypropylene syringes with piston plugs made of a conventional rubber material, it behaves in such a way that after the vapor sterilization has been carried out, the oxybutynin content drops to 85 to 90% of the previously present active ingredient content. During vapor sterilization in piston syringes made of polypropylene with piston plugs made of a conventional rubber material the oxybutynin decomposition product phenylcyclohexylglycolic acid is formed, and in such quantities that are a factor of 2 to 3 above the specification limit. In addition, further impurities result in quantities sometimes exceeding 0.5%.

Without being bound to any theory, the acid resistance sometimes is—in contrast to the materials used according to the present invention—very low for polypropylene, wherein the high temperatures during vapor sterilization also promote possible reactions between the composition and the syringe material, which can also lead to the formation of further impurities. In this context, the degradation product of oxybutynin hydrochloride in the form of phenylcyclohexylglycolic acid can particularly be produced by two different reactions, namely saponification in alkaline solution on the one hand and hydrolysis in acidic solution on the other hand. Since the pH value of the composition is in the acidic range, it can be assumed—without being bound to this theory—that the degradation product is primarily the result of a hydrolysis of oxybutynin hydrochloride, wherein this reaction is further intensified by the high temperatures present during sterilization. The hydrolysis of the active ingredient results in both a reduction of the active ingredient content and the formation of phenylcyclohexylglycolic acid during vapor sterilization. Furthermore, in contrast to the present invention, the formation of phenylcyclohexylglycolic acid is further enhanced or accelerated as a result of the rubber material normally used for the piston plug. Without limiting this theory, it is generally the case that rubber materials commonly used for piston plugs generally comprise a high proportion of unsaturated C—C double bonds, which results in an increased reactivity of the material. In addition, and without limiting this theory, phenylcyclohexylglycolic acid is more reactive than the active ingredient due to the free acid group, so that phenylcyclohexylglycolic acid formed during sterilization reacts at least partially with the rubber material and is thus removed from the reaction equilibrium, which favors the degradation of the active ingredient.

In contrast, the present invention uses piston syringes based on cycloolefin-(co)polymers (COP or COC) and piston plugs or closing caps based on halobutyl rubber, especially chlorobutyl rubber or bromobutyl rubber, preferably bromobutyl rubber, and a respective oxybutynin-comprising composition, a significantly improved stability of the composition is observed with a lower degradation of the active ingredient and thus a reduced formation of degradation products, especially phenylcyclohexylglycolic acid, under defined vapor sterilization. In contrast to polypropylene, the cycloolefin-(co)polymer (COP or COC) used for the piston syringe according to the invention has a high stability even against acidic compositions. In addition, it behaves in relation to the piston plug or the closing cap in such a way that the halobutyl rubber used for this purpose according to the invention comprises a considerably lower number of C—C double bonds and is therefore less reactive than rubber materials normally used. In addition, halobutyl rubber comprises improved heat stability and improved aging properties and low gas permeability.

On this basis, within the scope of the present invention, a respective sterile oxybutynin-comprising composition with improved properties can be obtained by vapor sterilization, both with respect to the guarantee of a high sterility and with respect to the guarantee of a high or unchanged active ingredient content, due to a significant reduction of the formation of corresponding degradation products.

Overall, it can thus be stated that, on the basis of the method according to the invention, using a special oxybutynin-comprising composition and using a special piston syringe with special components in this respect, an efficient and at the same time gentle vapor sterilization of the underlying composition can be carried out, wherein the degradation of the active ingredient is reduced, so that, in this respect, corresponding stable compositions are also provided.

The present invention will be better understood by the following aspects, which form part of the present invention:

### Aspect 1:

1. A method for producing a sterile oxybutynin-comprising composition in a preferably ready-to-use piston syringe, especially a disposable piston syringe, and/or for producing a preferably ready-to-use sterile piston syringe filled with an oxybutynin-comprising composition, especially a disposable piston syringe,
   wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber;
   wherein the method comprises the following steps:
   a) filling the piston syringe with the oxybutynin-comprising composition and/or providing the oxybutynin-comprising composition in the piston syringe,
      wherein the oxybutynin is used in the form of the hydrochloride, the oxybutynin-comprising composition is configured as an aqueous composition and the oxybutynin-comprising composition comprises, in combination and in each case in effective, especially pharmaceutically effective, amounts,
         oxybutynin hydrochloride (oxybutynin-HCl) in a concentration of (1±0.5) mg/ml and
         sodium chloride, calculated as sodium, in a concentration of (3.5±0.5) mg/ml,
      wherein the oxybutynin-comprising composition is adjusted to a pH value in the range of from 3 to 5, especially in the range of from 3.8 to 4.5, and/or wherein the oxybutynin-comprising composition comprises a pH value in the range of from 3 to 5, especially in the range of from 3.8 to 4.5;
   and subsequently
   b) vapor sterilization, especially steam sterilization, of the oxybutynin-comprising composition in the piston syringe and/or of the piston syringe filled with the oxybutynin-comprising composition.

### Aspect 2:

2. The method according to aspect 1,
   wherein the oxybutynin-comprising composition comprises the oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml.

### Aspect 3:

3. The method according to aspect 1 or 2,
   wherein the oxybutynin-comprising composition comprises the sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml.

### Aspect 4:

4. The method according to any of the preceding aspects,
   wherein the pH value of the oxybutynin-comprising composition is adjusted using and/or by means of hydrochloric acid.

### Aspect 5:

5. The method according to any of the preceding aspects,
   wherein the piston syringe has a volume, especially a receiving volume for the oxybutynin-comprising composition, in the range of from 4 ml to 25 ml, especially in the range of from 6 ml to 20 ml, preferably in the range of from 8 ml to 15 ml, preferably in the range of from 9 ml to 12 ml, more preferably of about 10 ml; and/or
   wherein the piston syringe comprises the oxybutynin-comprising composition in an amount in the range of from 4 ml to 25 ml, especially in the range of from 6 ml to 20 ml, preferably in the range of from 8 ml to 15 ml, preferably in the range of from 9 ml to 12 ml, more preferably of about 10 ml.

### Aspect 6:

6. The method according to any of the preceding aspects,
   wherein the piston plug is formed from a chlorobutyl rubber or from a bromobutyl rubber, preferably from a bromobutyl rubber.

### Aspect 7:

7. The method according to any of the preceding aspects,
   wherein the piston syringe, especially the syringe cylinder, comprises a first opening, especially for receiving a syringe piston; and/or
   wherein the piston syringe comprises a syringe piston, especially wherein the syringe piston is formed from polypropylene or especially wherein the syringe piston is formed from a cycloolefin-(co)polymer (COP or COC).

### Aspect 8:

8. The method according to any of the preceding aspects,
   wherein the piston syringe comprises a second opening, especially a discharge opening, preferably for discharging and/or applying the oxybutynin-comprising composition.

### Aspect 9:

9. The method according to any of the preceding aspects,
   wherein the piston syringe comprises a closing element, especially a closing cap, preferably for closing the second opening, especially wherein the closing element is formed from a halobutyl rubber, especially from a chlorobutyl rubber or from a bromobutyl rubber, preferably from a bromobutyl rubber.

### Aspect 10:

10. The method according to any of the preceding aspects,
   wherein vapor sterilization is carried out under the application of temperature (heating); and/or
   wherein vapor sterilization is carried out at temperatures of at least 105° C., especially of at least 110° C., preferably of at least 115° C., preferably of at least 120° C.; and/or
   wherein vapor sterilization is carried out at temperatures of at most 160° C., especially of at most 150° C., preferably of at most 140° C., preferably of at most 130° C.; and/or
   wherein vapor sterilization is carried out at temperatures in the range of from 105° C. to 160° C., especially in the range of from 110° C. to 150° C., preferably in the range of from 115° C. to 140° C., preferably in the range of from 20° C. to 130° C., more preferably of about 121° C.

### Aspect 11:

11. The method according to any of the preceding aspects,
   wherein vapor sterilization is carried out for a duration of at least 1 min, especially of at least 2 min, preferably of at least 3 min, preferably of at least 5 min, more preferably of at least 8 min, most preferably of at least 10 min, even more preferably of at least 15 min, yet even more preferably of at least 17 min; and/or
   wherein vapor sterilization is carried out for a duration of at most 500 min, especially of at most 400 min, preferably of at most 200 min, preferably of at most 100 min, more preferably of at most 60 min, most preferably of at most 45 min, even more preferably of at most 30 min, yet even more preferably of at most 25 min; and/or
   wherein vapor sterilization is carried out for a duration in the range of from 1 min to 500 min, especially in the range of from 2 min to 400 min, preferably in the range of from 3 min to 200 min, preferably in the range of from 5 min to 100 min, more preferably in the range of from 8 min to 60 min, most preferably in the range of from 10 min to 45 min, even more preferably in the range of from 15 min to 30 min, yet even more preferably in the range of from 17 min to 25 min, yet again more preferably of about 20 min.

### Aspect 12:

12. The method according to any of the preceding aspects,
   wherein vapor sterilization is carried out under pressurization.

### Aspect 13:

13. The method according to any of the preceding aspects,
   wherein vapor sterilization is carried out at a relative pressure in the range of from 0.1 bar to 10 bar, especially in the range of from 0.5 bar to 5 bar, preferably in the range of from 1 bar to 3.5 bar, preferably in the range of from 1.5 bar to 3 bar; and/or
   wherein vapor sterilization is carried out at an absolute pressure in the range of from 1.1 bar to 10 bar, especially in the range of from 1.5 bar to 6 bar, preferably in the range of from 2 bar to 4.5 bar.

### Aspect 14:

14. The method according to any of the preceding aspects,
   wherein vapor sterilization is carried out in the presence of an atmosphere comprising especially pure steam and/or as a saturated steam process; or wherein vapor sterilization is carried out in the presence of a steam/gas mixture, especially a mixture of steam with inert gas, especially nitrogen, and/or oxygen, preferably a steam/air mixture.

### Aspect 15:

15. The method according to any of the preceding aspects,
   wherein vapor sterilization is carried out in a preferably closed sterilization device, especially in a gas-tightly closed pressure vessel, preferably in an autoclave apparatus (autoclave);
   especially wherein the sterilization atmosphere is generated by injecting steam and/or steam/compressed air mixtures, preferably steam/compressed air mixtures, into the sterilization device; and/or
   especially wherein the sterilization atmosphere is generated by injection of already premixed steam/compressed air mixtures and/or by separate injections of steam and compressed air into the sterilization device.

### Aspect 16:

16. The method according to any of the preceding aspects,
   wherein, during vapor sterilization, steam is generated from distilled and/or demineralized water; and/or
   wherein, during vapor sterilization, compressed air is generated from sterile air, especially wherein the air is cleaned and/or sterilized by passing through filters.

### Aspect 17:

17. The method according to any of the preceding aspects,
   wherein vapor sterilization is carried out under overkill conditions and/or at about 121° C. and/or for a duration of at least 15 min, especially of at least 20 min, preferably of about 20 min; and/or
   wherein vapor sterilization is carried out according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

### Aspect 18:

18. The method according to any of the preceding aspects,
   wherein vapor sterilization is carried out multi-cyclically, especially wherein at least the final and/or terminal cycle is carried out under at least one of the conditions defined in Aspects 10 to 17 and/or especially wherein at least the final and/or terminal cycle is carried out under overkill conditions and/or at about 121° C. and/or for a duration of at least 15 min, especially of at least 20 min, preferably of about 20 min.

### Aspect 19:

19. The method according to any of the preceding aspects,
   wherein the piston syringe filled with the oxybutynin-comprising composition has been inserted, especially prior to the performance of the vapor sterilization, into a packaging, especially a stand-by packaging, which is closed and/or sealed, especially in a water- and/or germ-proof manner, especially wherein at least part of the packaging is configured to be at least steam-permeable, especially steam- and/or gas-permeable, especially wherein the packaging is also sterilized when the vapor sterilization is performed.

### Aspect 20:

20. The method according to any of the preceding aspects,
   wherein a plurality of piston syringes filled with the oxybutynin-comprising composition is sterilized simultaneously, especially wherein preferably, during the performance of the vapor sterilization, several piston syringes are brought together on a (syringe) carrier.

### Aspect 21:

21. The method according to any of the preceding aspects,
   wherein the sterile oxybutynin-comprising composition obtained after vapor sterilization and/or the sterile piston syringe filled with the oxybutynin-comprising composition obtained after vapor sterilization has a SAL (Sterility Assurance Level) value of at least 105, especially of at least 10⁻⁶, preferably of at least 10⁻⁷.

### Aspect 22:

22. The method according to any of the preceding aspects,
   wherein the sterile oxybutynin-comprising composition obtained after vapor sterilization has a content of degradation product(s) of oxybutynin, especially a content of phenylcyclohexylglycolic acid, of at most 10% by weight, especially of at most 7.5% by weight, preferably of at most 5% by weight, preferably of at most 2% by weight, more preferably of at most 1% by weight, based on oxybutynin hydrochloride.

### Aspect 23:

23. A method for producing a sterile oxybutynin-comprising composition in a preferably ready-to-use piston syringe, especially a disposable piston syringe, and/or for producing a preferably ready-to-use, sterile piston syringe filled with an oxybutynin-comprising composition, especially a disposable piston syringe, especially a method according to any of the preceding aspects,
   wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber;
   wherein the method comprises the following steps:
   a) filling the piston syringe with the oxybutynin-comprising composition and/or providing the oxybutynin-comprising composition in the piston syringe,
      wherein the oxybutynin is used in the form of the hydrochloride, the oxybutynin-comprising composition is configured as an aqueous composition and the oxybutynin-comprising composition comprises, in combination and in each case in effective, especially pharmaceutically effective, amounts,
         oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml, and
         sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml,
      wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5;
   and subsequently
   b) vapor sterilization, especially steam sterilization, of the oxybutynin-comprising composition in the piston syringe and/or of the piston syringe filled with the oxybutynin-comprising composition.

### Aspect 24:

24. A method for producing a sterile oxybutynin-comprising composition in a preferably ready-to-use piston syringe, especially a disposable piston syringe, and/or for producing a preferably ready-to-use sterile piston syringe filled with an oxybutynin-comprising composition, especially a disposable piston syringe, especially a method according to any of the preceding aspects,
   wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber;
   wherein the method comprises the following steps:
   a) filling the piston syringe with the oxybutynin-comprising composition and/or providing the oxybutynin-comprising composition in the piston syringe,
      wherein the oxybutynin is used in the form of the hydrochloride, the oxybutynin-comprising composition is configured as an aqueous composition and the oxybutynin-comprising composition comprises, in combination and in each case in effective, especially pharmaceutically effective, amounts,
         oxybutynin hydrochloride (oxybutynin-HCl) in a concentration of about 1 mg/ml and
         sodium chloride, calculated as sodium, in a concentration of about 3.5 mg/ml,
      wherein the oxybutynin-comprising composition is adjusted to a pH value in the range of from 3.8 to 4.5 and/or wherein the oxybutynin-comprising composition has a pH value in the range of from 3.8 to 4.5;
   and subsequently
   b) vapor sterilization, especially steam sterilization, of the oxybutynin-comprising composition in the piston syringe and/or of the piston syringe filled with the oxybutynin-comprising composition.

### Aspect 25:

25. A method for producing a sterile oxybutynin-comprising composition in a preferably ready-to-use piston syringe, especially a disposable piston syringe, and/or for producing a preferably ready-to-use sterile piston syringe filled with an oxybutynin-comprising composition, especially a disposable piston syringe, especially method according to any of the preceding aspects,
   wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber;
   wherein the method comprises the following steps:
   a) filling the piston syringe with the oxybutynin-comprising composition and/or providing the oxybutynin-comprising composition in the piston syringe,
      wherein the oxybutynin is used in the form of the hydrochloride, the oxybutynin-comprising composition is configured as an aqueous composition and the oxybutynin-comprising composition comprises, in combination and in each case in effective, especially pharmaceutically effective, amounts,
         oxybutynin hydrochloride (oxybutynin-HCl) in a concentration of about 1 mg/ml and
         sodium chloride, calculated as sodium, in a concentration of about 3.5 mg/ml,
      wherein the oxybutynin-comprising composition is adjusted to a pH value in the range of from 3.8 to 4.5 and/or wherein the oxybutynin-comprising composition has a pH value in the range of from 3.8 to 4.5;
   and subsequently
   b) vapor sterilization, especially steam sterilization, of the oxybutynin-comprising composition in the piston syringe and/or of the piston syringe filled with the oxybutynin-comprising composition,
      wherein the vapor sterilization is carried out under overkill conditions and/or at about 121° C. and/or for a duration of at least 15 min, especially of at least 20 min, preferably of about 20 min, and/or
      wherein the vapor sterilization is carried out according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

### Aspect 26:

26. A sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
   wherein the oxybutynin-comprising composition is obtainable by a method according to any of Aspects 1 to 25; and/or
   wherein the oxybutynin-comprising composition has been vapor sterilized, especially steam-sterilized, by a method according to any of Aspects 1 to 25.

### Aspect 27:

27. A sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy, especially the composition according to aspect 26,
   wherein the oxybutynin-comprising composition is present in a preferably ready-to-use piston syringe, especially a disposable piston syringe,
   wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber;
   wherein the oxybutynin-comprising composition comprises the oxybutynin in the form of the hydrochloride, the oxybutynin-comprising composition is configured as an aqueous composition and the oxybutynin-comprising composition comprises, in combination and in each case in effective, especially pharmaceutically effective, amounts,
      oxybutynin hydrochloride (oxybutynin-HCl) in a concentration of (1±0.5) mg/ml and
      sodium chloride, calculated as sodium, in a concentration of (3.5±0.5) mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the oxybutynin-comprising composition comprised in the piston syringe, including the piston syringe, is vapor sterilized, especially steam-sterilized.

### Aspect 28:

28. The composition according to aspect 26 or 27,
   wherein the oxybutynin-comprising composition comprises the oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml; and/or
   wherein the oxybutynin-comprising composition comprises the sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml.

### Aspect 29:

29. The composition according to any of aspects 26 to 28,
   wherein the piston plug is formed from a chlorobutyl rubber or from a bromobutyl rubber, preferably from a bromobutyl rubber.

### Aspect 30:

30. The composition according to any of aspects 26 to 29,
   wherein the oxybutynin-comprising composition is vapor sterilized under at least one of the conditions defined in Aspects 10 to 18; and/or
   wherein the oxybutynin-comprising composition is vapor sterilized under overkill conditions and/or at about 121° C. and/or for a duration of at least 15 min, especially of at least 20 min, preferably of about 20 min; and/or
   wherein the oxybutynin-comprising composition is vapor sterilized according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

### Aspect 31:

31. The composition according to any of aspects 26 to 30,
   wherein the sterile oxybutynin-comprising composition obtained after vapor sterilization and/or the sterile piston syringe filled with the oxybutynin-comprising composition obtained after vapor sterilization has a SAL (Sterility Assurance) value of at least 10⁻⁵, especially of at least 10⁻⁶, preferably of at least 10⁻⁷.

### Aspect 32:

32. The composition according to any of aspects 26 to 31,
   wherein the sterile oxybutynin-comprising composition obtained after vapor sterilization has a content of degradation product(s) of oxybutynin, especially a content of phenylcyclohexylglycolic acid, of at most 10% by weight, especially of at most 7.5% by weight, preferably of at most 5% by weight, preferably of at most 2% by weight, more preferably of at most 1% by weight, based on oxybutynin hydrochloride.

### Aspect 33:

33. The composition according to any of aspects 26 to 32,
   wherein the neurogenic bladder dysfunctions are associated with and/or characterized by detrusor overactivity and/or detrusor-sphincter dyssynergia, especially overactive detrusor; and/or
   herein the neurogenic bladder dysfunctions are associated with or caused by spinal cord injury, especially paraplegia, spina bifida, diabetes, multiple sclerosis, stroke or Parkinson's disease.

### Aspect 34:

34. A sterile oxybutynin-comprising composition, especially for use in the prophylactic and/or therapeutic treatment of neurogenic bladder dysfunction, especially neurogenic voiding disorders, preferably neurogenic bladder dysfunction associated with detrusor hyperactivity and/or detrusor sphincter dyssynergy, especially composition according to any of aspects 26 to 33,
   wherein the oxybutynin-comprising composition is present in a preferably ready-to-use piston syringe, especially a disposable piston syringe,
   wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or bromobutyl rubber, preferably from a bromobutyl rubber;
   wherein the oxybutynin-comprising composition comprises the oxybutynin in the form of the hydrochloride, the oxybutynin-comprising composition is configured as an aqueous composition and the oxybutynin-comprising composition comprises, in combination and in each case in effective, especially pharmaceutically effective, amounts,
      oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml, and
      sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the oxybutynin-comprising composition comprised in the piston syringe, including the piston syringe, is vapor sterilized, especially steam-sterilized.

### Aspect 35:

35. A sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy, especially composition according to any of aspects 26 to 34,
   wherein the oxybutynin-comprising composition is present in a preferably ready-to-use piston syringe, especially a disposable piston syringe,
   wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber;
   wherein the oxybutynin-comprising composition comprises the oxybutynin in the form of the hydrochloride, the oxybutynin-comprising composition is configured as an aqueous composition and the oxybutynin-comprising composition comprises, in combination and in each case in effective, especially pharmaceutically effective, amounts,
      oxybutynin hydrochloride (oxybutynin-HCl) in a concentration of about 1 mg/ml and
      sodium chloride, calculated as sodium, in a concentration of about 3.5 mg/ml,
   wherein the oxybutynin-comprising composition is adjusted to a pH value in the range of from 3.8 to 4.5, and/or wherein the oxybutynin-comprising composition has a pH value in the range of from 3.8 to 4.5; and
   wherein the oxybutynin-comprising composition comprised in the piston syringe, including the piston syringe, is vapor sterilized, especially steam-sterilized.

### Aspect 36:

36. A sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy, especially composition according to any of aspects 26 to 35,
   wherein the oxybutynin-comprising composition is present in a preferably ready-to-use piston syringe, especially a disposable piston syringe,
   wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber;
   wherein the oxybutynin-comprising composition comprises the oxybutynin in the form of the hydrochloride, the oxybutynin-comprising composition is configured as an aqueous composition and the oxybutynin-comprising composition comprises, in combination and in each case in effective, especially pharmaceutically effective, amounts,
      oxybutynin hydrochloride (oxybutynin-HCl) in a concentration of about 1 mg/ml and
      sodium chloride, calculated as sodium, in a concentration of about 3.5 mg/ml,
   wherein the oxybutynin-comprising composition is adjusted to a pH value in the range of from 3.8 to 4.5, and/or wherein the oxybutynin-comprising composition has a pH value in the range of from 3.8 to 4.5; and
   wherein the oxybutynin-comprising composition comprised in the piston syringe, including the piston syringe, is vapor sterilized, especially steam-sterilized,
   wherein the vapor sterilization is carried out under overkill conditions and/or at about 121° C. and/or for a duration of at least 15 min, especially of at least 20 min, preferably of about 20 min, and/or
   wherein the vapor sterilization is carried out according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

### Aspect 37:

37. The composition according to any of aspects 26 to 36,
   for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
   especially wherein the neurogenic bladder dysfunction is associated with and/or characterized by detrusor overactivity and/or detrusor-sphincter dyssynergia, especially overactive detrusor; and/or
   especially wherein the neurogenic bladder dysfunction is associated with or caused by a spinal cord injury, especially paraplegia, spina bifida, diabetes, multiple sclerosis, stroke or Parkinson's disease; and/or
   for instillation and/or for preferred topical application into the urogenital region, especially into the bladder; and/or
   wherein the oxybutynin-comprising composition is prepared for administration and/or for instillation and/or for preferably topical application into the urogenital tract, especially into the bladder, and/or wherein the oxybutynin-comprising composition is administered by instillation and/or for preferably topical application into the urogenital tract, especially into the bladder.

### Aspect 38:

38. A piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber,
   wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy, especially as defined in any of Aspects 26 to 37,
   wherein the piston syringe is obtainable by a method according to any of Aspects 1 to 25; and/or
   wherein the oxybutynin-comprising composition comprised in the piston syringe, including the piston syringe, has been vapor sterilized, especially steam-sterilized, by a method according to any of Aspects 1 to 25.

### Aspect 39:

39. A piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially piston syringe according to aspect 38,
   wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergia,
   wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
      oxybutynin hydrochloride (oxybutynin-HCl) in a concentration of (1±0.5) mg/ml and
      sodium chloride, calculated as sodium, in a concentration of (3.5±0.5) mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the oxybutynin-comprising composition comprised in the piston syringe, including the piston syringe, is vapor sterilized, especially steam-sterilized.

### Aspect 40:

40. The piston syringe according to aspect 38 or 39,
   wherein the oxybutynin-comprising composition comprises the oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml.

### Aspect 41:

41. The piston syringe according to any of aspects 38 to 40,
   wherein the oxybutynin-comprising composition comprises the sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml.

### Aspect 42:

42. The piston syringe according to any of aspects 38 to 41,
   wherein the piston syringe has a volume, especially a receiving volume for the oxybutynin-comprising composition, in the range of from 4 ml to 25 ml, especially in the
   range of from 6 ml to 20 ml, preferably in the range of from 8 ml to 15 ml, preferably in the range of from 9 ml to 12 ml, more preferably of about 10 ml; and/or
   wherein the piston syringe comprises the oxybutynin-comprising composition in an amount in the range of from 4 ml to 25 ml, especially in the range of from 6 ml to 20 ml, preferably in the range of from 8 ml to 15 ml, preferably in the range of from 9 ml to 12 ml, more preferably of about 10 ml.

### Aspect 43:

43. The piston syringe according to any of aspects 38 to 42,
   wherein the piston plug is formed from a chlorobutyl rubber or from a bromobutyl rubber, preferably from a bromobutyl rubber.

### Aspect 44:

44. The piston syringe according to any of aspects 38 to 43,
   wherein the piston syringe, especially the syringe cylinder, comprises a first opening, especially for receiving a syringe piston; and/or
   wherein the piston syringe comprises a syringe piston, especially wherein the syringe piston is formed from polypropylene or especially wherein the syringe piston is formed from a cycloolefin-(co)polymer (COP or COC).

### Aspect 45:

45. The piston syringe according to any of aspects 38 to 44,
   wherein the piston syringe comprises a second opening, especially a discharge opening, preferably for discharging and/or applying the oxybutynin-comprising composition.

### Aspect 46:

46. The piston syringe according to any of aspects 38 to 45,
   wherein the piston syringe comprises a closing element, especially a closing cap, preferably for closing the second opening, especially wherein the closing element is formed from a halobutyl rubber, especially from a chlorobutyl rubber or from a bromobutyl rubber, preferably from a bromobutyl rubber.

### Aspect 47:

47. The piston syringe according to any of aspects 38 to 46,
   wherein vapor sterilization is carried out under at least one of the conditions defined in Aspects 10 to 18; and/or
   wherein vapor sterilization is carried out under overkill conditions and/or at about 121° C. and/or for a duration of at least 15 min, especially of at least 20 min, preferably of about 20 min; and/or
   wherein vapor sterilization is carried out according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

### Aspect 48:

48. The piston syringe according to any of aspects 38 to 47,
   wherein the sterile piston syringe filled with the sterile oxybutynin-comprising composition obtained after vapor sterilization and/or the oxybutynin-comprising composition obtained after vapor sterilization comprises a Sterility Assurance (SAL) value of at least 10⁻⁵, especially of at least 10⁻⁶, preferably of at least 10⁻⁷; and/or
   for instillation and/or for the preferred topical application of the oxybutynin-comprising composition into the urogenital region, especially into the bladder.

### Aspect 49:

49. A piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber, especially piston syringe according to any of aspects 38 to 48,
   wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
   wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
      oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml, and
      sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the oxybutynin-comprising composition comprised in the piston syringe, including the piston syringe, is vapor sterilized, especially steam-sterilized.

### Aspect 50:

50. A piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber, especially piston syringe according to any of aspects 38 to 49,
   wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
   wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
      oxybutynin hydrochloride (oxybutynin-HCl) in a concentration of (1±0.4) mg/ml, especially in a concentration of (1±0.3) mg/ml, preferably in a concentration of (1±0.2) mg/ml, preferably in a concentration of (1±0.1) mg/ml, more preferably in a concentration of about 1 mg/ml, and
      sodium chloride, calculated as sodium, in a concentration of (3.5±0.4) mg/ml, especially in a concentration of (3.5±0.3) mg/ml, preferably in a concentration of (3.5±0.2) mg/ml, preferably in a concentration of (3.5±0.1) mg/ml, more preferably in a concentration of about 3.5 mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the oxybutynin-comprising composition comprised in the piston syringe, including the piston syringe, is vapor sterilized, especially steam-sterilized.

### Aspect 51:

51. A piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber, especially piston syringe according to any of aspects 38 to 50,
   wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
   wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
      oxybutynin hydrochloride (oxybutynin-HCl) in a concentration of about 1 mg/ml and
      sodium chloride, calculated as sodium, in a concentration of about 3.5 mg/ml,
   wherein the oxybutynin-comprising composition is adjusted to a pH value in the range of from 3.8 to 4.5 and/or wherein the oxybutynin-comprising composition has a pH value in the range of from 3.8 to 4.5; and
   wherein the oxybutynin-comprising composition comprised in the piston syringe, including the piston syringe, is vapor sterilized, especially steam-sterilized.

### Aspect 52:

52. A piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber, especially piston syringe according to any of aspects 38 to 51,
   wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
   wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
      oxybutynin hydrochloride (oxybutynin-HCl) in a concentration of about 1 mg/ml and
      sodium chloride, calculated as sodium, in a concentration of about 3.5 mg/ml,
   wherein the oxybutynin-comprising composition is adjusted to a pH value in the range of from 3.8 to 4.5 and/or wherein the oxybutynin-comprising composition has a pH value in the range of from 3.8 to 4.5; and
   wherein the oxybutynin-comprising composition comprised in the piston syringe, including the piston syringe, is vapor sterilized, especially steam-sterilized,
   wherein the vapor sterilization is carried out under overkill conditions and/or at about 121° C. and/or for a duration of at least 15 min, especially of at least 20 min, preferably of about 20 min, and/or
   wherein the vapor sterilization is carried out according to Pharmacopoea Europaea (Ph. Eur.), chapter 5.1.1 (07/2017:50101), especially under overkill conditions and/or at about 121° C. and/or for a duration of at least 15 min, especially of at least 20 min, preferably of about 20 min.

### Aspect 53:

53. A piston syringe, especially a disposable piston syringe, wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber, especially from a chlorobutyl rubber or from a bromobutyl rubber, preferably from a bromobutyl rubber, especially piston syringe according to any of aspects 38 to 52,
   wherein the piston syringe is filled with a sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergy,
   wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
      oxybutynin hydrochloride (oxybutynin-HCl) in a concentration of about 1 mg/ml and
      sodium chloride, calculated as sodium, in a concentration of about 3.5 mg/ml,
   wherein the oxybutynin-comprising composition is adjusted to a pH value in the range of from 3.8 to 4.5 and/or wherein the oxybutynin-comprising composition has a pH in the range of from 3.8 to 4.5; and
   wherein the piston syringe comprises a volume, especially a receiving volume for the oxybutynin-comprising composition, of about 10 ml, and/or wherein the piston syringe comprises the oxybutynin-comprising composition in an amount of about 10 ml,
   wherein the oxybutynin-comprising composition comprised in the piston syringe, including the piston syringe, is vapor sterilized, especially steam-sterilized.

### Aspect 54:

54. A packaging unit comprising at least one packaging and at least one piston syringe, especially disposable piston syringe, as defined in one of Aspects 38 to 53, wherein the piston syringe is inserted into the packaging and/or is present in the packaging.

### Aspect 55:

55. A kit, especially an application and/or instillation system, comprising (i) at least one oxybutynin-comprising composition, as defined in one of Aspects 26 to 37, present in a piston syringe, especially disposable piston syringe, especially as defined in one of Aspects 38 to 53; (ii) (a) at least one application and/or instillation device connectable to the piston syringe, especially in the form of an instillation tube or the like, preferably in the form of a (urinary bladder) catheter, and/or (b) at least one connection device, especially an adapter, connectable to the piston syringe for connecting to an application and/or instillation device, especially in the form of an instillation tube or the like, preferably in the form of a (urinary bladder) catheter; and optionally (iii) at least one application and/or instillation manual.

### Aspect 56:

56. A kit, especially an application and/or instillation system, comprising (i) at least one piston syringe, especially a disposable piston syringe, as defined in one of Aspects 38 to 53, and/or at least one packaging unit as defined in Aspect 54; (ii) (a) at least one application and/or instillation device connectable to the piston syringe, especially in the form of an instillation tube or the like, preferably in the form of a (urinary bladder) catheter, and/or (b) at least one connecting device, especially an adapter, connectable to the piston syringe for connecting to an application and/or instillation device, especially in the form of an instillation tube or the like, preferably in the form of a (urinary bladder) catheter; and optionally (iii) at least one application and/or instillation manual.

### Aspect 57:

57. Use of a piston syringe, especially a disposable piston syringe, in a method for producing a sterile oxybutynin-comprising composition, especially as defined in one of Aspects 1 to 25,
   wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber,
   wherein the piston syringe is filled with the sterile oxybutynin-comprising composition, especially for use in the prophylactic and/or therapeutic treatment of neurogenic bladder dysfunction, especially neurogenic bladder voiding disorders, preferably neurogenic bladder dysfunction associated with detrusor hyperactivity and/or detrusor sphincter dyssynergia,
   wherein the oxybutynin-comprising composition comprises the oxybutynin in the form of the hydrochloride, the oxybutynin-comprising composition is configured as an aqueous composition and the oxybutynin-comprising composition comprises, in combination and each in effective, especially pharmaceutically effective, amounts,
      oxybutynin hydrochloride (oxybutynin-HCl) in a concentration of (1±0.5) mg/ml and
      sodium chloride, calculated as sodium, in a concentration of (3.5±0.5) mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the oxybutynin-comprising composition comprised in the piston syringe, including the piston syringe, is vapor sterilized, especially steam-sterilized.

### Aspect 58:

58. Use of vapor sterilization, especially steam sterilization, in a method for producing a sterile oxybutynin-comprising composition in a preferably ready-to-use piston syringe, especially disposable piston syringe, and/or for producing a preferably ready-to-use, sterile piston syringe filled with an oxybutynin-comprising composition, especially disposable piston syringe, especially as defined in one of aspects 1 to 25,
   wherein the piston syringe comprises a syringe body, especially a syringe cylinder, on the one hand and a syringe piston with a piston plug on the other hand, wherein the syringe body is formed from a cycloolefin-(co)polymer (COP or COC) and wherein the piston plug is formed from a halobutyl rubber;
   wherein in the oxybutynin-comprising composition the oxybutynin is used in the form of the hydrochloride, the oxybutynin-comprising composition is configured as an aqueous composition and the oxybutynin-comprising composition comprises, in combination and in each case in effective, especially pharmaceutically effective, amounts,
      oxybutynin hydrochloride (oxybutynin-HCl) in a concentration of (1±0.5) mg/ml and
      sodium chloride, calculated as sodium, in a concentration of (3.5±0.5) mg/ml,
   wherein the oxybutynin-comprising composition is adjusted to a pH value in the range of from 3 to 5, especially in the range of from 3.8 to 4.5, and/or wherein the oxybutynin-comprising composition has a pH value in the range of from 3 to 5, especially in the range of from 3.8 to 4.5; and
   wherein the piston syringe filled with the oxybutynin-comprising composition and/or the oxybutynin-comprising composition provided in the piston syringe is vapor sterilized, especially steam-sterilized.

### Aspect 59:

59. Piston syringe according to any of Aspects 38 to 53, packaging unit according to Aspect 54, kit according to Aspect 55 or 56, use according to Aspect 57 and/or use
   according to Aspect 58, each characterized by one or more of the features of Aspect 1 to 37.

All aspects of the present invention, as delineated hereinabove, form part of the present invention also in their respective combinations.

1. A method for producing a sterile oxybutynin-comprising composition in a ready-to-use piston syringe filled with the oxybutynin-comprising composition,
wherein the piston syringe comprises a syringe body in the form of a syringe cylinder, on the one hand, and a syringe piston with a piston plug, on the other hand, wherein the syringe body is formed from a cycloolefin-(co)polymer and wherein the piston plug is formed from a halobutyl rubber;
wherein the method comprises the following steps:
a) filling the piston syringe with the oxybutynin-comprising composition and providing the oxybutynin-comprising composition in the piston syringe,
wherein the oxybutynin is used in the form of the hydrochloride, wherein the oxybutynin-comprising composition is configured as an aqueous composition and wherein the oxybutynin-comprising composition comprises, in combination and in each case in pharmaceutically effective amounts:
oxybutynin hydrochloride in a concentration of (1±0.5) mg/ml and
sodium chloride, calculated as sodium, in a concentration of (3.5±0.5) mg/ml,
wherein the oxybutynin-comprising composition is adjusted to a pH value in the range of from 3 to 5;
and subsequently:
b) performing a vapor sterilization of the oxybutynin-comprising composition in the piston syringe and of the piston syringe filled with the oxybutynin-comprising composition.

2. The method according to claim 1,
wherein the oxybutynin-comprising composition comprises the oxybutynin hydrochloride in a concentration of (1±0.1) mg/ml;
wherein the oxybutynin-comprising composition comprises the sodium chloride, calculated as sodium, in a concentration of (3.5±0.1) mg/ml;
wherein the pH value of the oxybutynin-comprising composition is adjusted using hydrochloric acid.

3. The method according to claim 1,
wherein vapor sterilization is carried out as a steam sterilization for a duration in the range of from 1 min to 500 min at temperatures in the range of from 105° C. to 160° C. and under pressurization at an absolute pressure in the range of from 1.1 bar to 10 bar in the presence of an atmosphere comprising a mixture of steam with at least one gas selected among inert gases, oxygen, air and mixtures thereof in a closed autoclave apparatus.

4. The method according to claim 1,
wherein vapor sterilization is carried out under overkill conditions at about 121° C. and for a duration of at least 15 min.

5. The method according to claim 1,
wherein vapor sterilization is carried out according to the conditions of Pharmacopoea Europaea (Ph. Eur.), Chapter 5.1.1, Edition 07/2017: 50101, under overkill conditions and at about 121° C. and for a duration of at least 15 min.

6. The method according to claim 1,
wherein the piston syringe filled with the oxybutynin-comprising composition has been inserted, prior to the performance of the vapor sterilization, into a packaging closed or sealed in a water-proof and germ-proof manner, wherein at least part of the packaging is configured to be steam-permeable, wherein the packaging is also sterilized when the vapor sterilization is performed.

7. The method according to claim 1,
wherein a plurality of piston syringes filled with the oxybutynin-comprising composition is sterilized simultaneously, wherein during the performance of the vapor sterilization said plurality of piston syringes are brought together on a syringe carrier.

8. The method according to claim 1,
wherein the sterile oxybutynin-comprising composition obtained after vapor sterilization and the sterile piston syringe filled with the oxybutynin-comprising composition obtained after vapor sterilization have a SAL (Sterility Assurance Level) value of at least 10⁻⁵.

9. The method according to claim 1,
wherein the sterile oxybutynin-comprising composition obtained after vapor sterilization comprises a content of oxybutynin degradation products of at most 5% by weight, based on oxybutynin hydrochloride.

10. A method for producing a sterile oxybutynin-comprising composition in a ready-to-use piston syringe filled with the oxybutynin-comprising composition,
wherein the piston syringe comprises a syringe body in the form of a syringe cylinder, on the one hand, and a syringe piston with a piston plug, on the other hand, wherein the syringe body is formed from a cycloolefin-(co)polymer and wherein the piston plug is formed from a halobutyl rubber;
wherein the method comprises the following steps:
a) filling the piston syringe with the oxybutynin-comprising composition and providing the oxybutynin-comprising composition in the piston syringe,
wherein the oxybutynin is used in the form of the hydrochloride, the oxybutynin-comprising composition is configured as an aqueous composition and the oxybutynin-comprising composition comprises, in combination and in each case in pharmaceutically effective amounts:
oxybutynin hydrochloride (oxybutynin-HCl) in a concentration of about 1 mg/ml and
sodium chloride, calculated as sodium, in a concentration of about
wherein the oxybutynin-comprising composition is adjusted to a pH value in the range of from 3.8 to 4.5;
and subsequently:
b) performing a vapor sterilization as a steam sterilization of the oxybutynin-comprising composition in the piston syringe and of the piston syringe filled with the oxybutynin-comprising composition,
wherein the vapor sterilization is carried out under overkill conditions and at about 121° C. and for a duration of at least 15 min under the conditions of Pharmacopoea Europaea (Ph. Eur.), Chapter 5.1.1, Edition 07/2017: 50101.

11. A sterile oxybutynin-comprising composition to be used in the treatment of a neurogenic bladder dysfunction of a human patient,
wherein the oxybutynin-comprising composition is obtained by a method according to claim 1.

12. A sterile oxybutynin-comprising composition to be used in the treatment of a neurogenic bladder dysfunction of a human patient,
wherein the oxybutynin-comprising composition is present in a ready-to-use piston syringe,
wherein the piston syringe comprises a syringe body in the form of a syringe cylinder, on the one hand, and a syringe piston with a piston plug, on the other hand, wherein the syringe body is formed from a cycloolefin-(co)polymer and wherein the piston plug is formed from a halobutyl rubber;
wherein the oxybutynin-comprising composition comprises the oxybutynin in the form of the hydrochloride, wherein the oxybutynin-comprising composition is configured as an aqueous composition and wherein the oxybutynin-comprising composition comprises, in combination and in each case in pharmaceutically effective amounts:
oxybutynin hydrochloride in a concentration of (1±0.5) mg/ml and
sodium chloride, calculated as sodium, in a concentration of (3.5±0.5) mg/ml,
wherein the oxybutynin-comprising composition has a pH value in the range of from 3 to 5; and
wherein the oxybutynin-comprising composition comprised in the piston syringe, including the piston syringe, is vapor-sterilized via steam-sterilization.

13. The composition according to claim 12,
wherein the oxybutynin-comprising composition comprises the oxybutynin hydrochloride in a concentration of (1±0.1) mg/ml;
wherein the oxybutynin-comprising composition comprises the sodium chloride, calculated as sodium, in a concentration of (3.5±0.1) mg/ml;
wherein the pH value of the oxybutynin-comprising composition is adjusted using hydrochloric acid.

14. The composition according to claim 12,
wherein the sterile oxybutynin-comprising composition obtained after vapor sterilization and the sterile piston syringe filled with the oxybutynin-comprising composition obtained after vapor sterilization have a SAL (Sterility Assurance Level) value of at least 10⁻⁵.

15. The composition according to claim 12,
wherein the sterile oxybutynin-comprising composition obtained after vapor sterilization comprises a content of oxybutynin degradation products of at most 5% by weight, based on oxybutynin hydrochloride.

16. The composition according to claim 12,
wherein the sterile oxybutynin-comprising composition obtained after vapor sterilization comprises a content of oxybutynin degradation products of at most 1% by weight, based on oxybutynin hydrochloride.

17. A ready-to-use piston syringe,
wherein the piston syringe comprises a syringe body in the form of a syringe cylinder, on the one hand, and a syringe piston with a piston plug, on the other hand,
wherein the syringe body is formed from a cycloolefin-(co)polymer and wherein the piston plug is formed from a halobutyl rubber;
wherein the piston syringe is filled with a sterile oxybutynin-comprising composition, wherein the oxybutynin is used in the form of the hydrochloride, wherein the oxybutynin-comprising composition is configured as an aqueous composition and wherein the oxybutynin-comprising composition comprises, in combination and in each case in pharmaceutically effective amounts:
oxybutynin hydrochloride in a concentration of (1±0.5) mg/ml and
sodium chloride, calculated as sodium, in a concentration of (3.5±0.5) mg/ml,
wherein the oxybutynin-comprising composition is adjusted to a pH value in the range of from 3 to 5;
wherein the oxybutynin-comprising composition comprised in the piston syringe, including the piston syringe, has been vapor sterilized by steam-sterilization.

18. The ready-to-use piston syringe of claim 17,
wherein the piston syringe is configured and adapted to be used in the treatment of a neurogenic bladder dysfunction of a human patient.

19. The ready-to-use piston syringe of claim 17,
wherein the piston syringe has a volume in the range of from 4 ml to 25 ml; and
wherein the piston syringe comprises the oxybutynin-comprising composition in an amount in the range of from 4 ml to 25 ml.

20. The ready-to-use piston syringe of claim 17,
wherein the piston syringe comprises a first opening for receiving a syringe piston and wherein the piston syringe comprises a syringe piston formed from a polypropylene or from a cycloolefin-(co)polymer, and
wherein the piston syringe comprises a second opening for discharging and applying the oxybutynin-comprising composition, wherein the piston syringe comprises a closing cap for closing the second opening, wherein the closing cap is formed from a halobutyl rubber.

21. The ready-to-use piston syringe of claim 17,
wherein vapor sterilization is carried out under overkill conditions and at about 121° C. and for a duration of at least 15 min.

22. The ready-to-use piston syringe of claim 17,
wherein vapor sterilization is carried out under the conditions according to Pharmacopoea Europaea (Ph. Eur.), Chapter 5.1.1, Edition 07/2017: 50101, under overkill conditions and at about 121° C. and for a duration of at least 15 min.

23. The ready-to-use piston syringe of claim 17,
wherein the sterile piston syringe filled with the sterile oxybutynin-comprising composition obtained after vapor sterilization and the oxybutynin-comprising composition obtained after vapor sterilization comprise a Sterility Assurance (SAL) value of at least 10⁻⁵.

24. A kit comprising an instillation system, with the kit comprising:
(i) at least one sterile oxybutynin-comprising composition as defined in claim 12 and present in a ready-to-use piston syringe as defined in claim 17;
(ii) at least one instillation device connectable to the piston syringe; and
(iii) at least one instillation manual comprising application instructions.
